(19) United States
(12) Patent Application Publication
(10) Pub. No.: US 2026/0102073 A1
(43) Pub. Date: Apr. 16, 2026

(54) METHOD AND SYSTEM FOR MICROWAVE SCANNING OF BIOLOGICAL TISSUE

(71) Applicant: WAVE VIEW IMAGING INC., Calgary (CA)

(72) Inventors: Jeremie BOURQUI, Calgary (CA); Elise FEAR, Calgary (CA)

(21) Appl. No.: 19/115,176

(22) PCT Filed: Dec. 20, 2023
(86) PCT No.: PCT/CA2023/051721

§ 371 Date: Mar. 25, 2025

Related U.S. Application Data
(60) Provisional application No. 63/433,894, filed on Dec. 20, 2022.

(51) Int. Cl. A61B 5/0507; A61B 5/00

(52) U.S. Cl. CPC A61B 5/0507; A61B 5/7235; A61B 5/4312; A61B 2562/0228; A61B 2562/046

(57) ABSTRACT

Disclosed are systems and methods of microwave scanning of biological tissue, which enable the measurement of biological tissue response to microwave signals without other unwanted signals, hence removing the need for suboptimal filtering techniques. The collected signals may be conditioned using novel techniques. Also disclosed are systems and methods of image reconstruction of biological tissues, which provide quantitative images related to the electrical properties of the tissues with a repeatable and stable technique.

### CROSS-REFERENCE TO RELATED APPLICATIONS

This application claims the benefit of priority to U.S. Provisional Application No. 63/433,894 filed on Dec. 20, 2022, the entire contents of which are incorporated herein by reference in its entirety.

### FIELD

The present invention relates generally to scanning of biological tissues, and in particular, to a method and system for microwave scanning of biological tissue.

### BACKGROUND

Imaging and sensing of biological tissues with different technologies is desired to improve detection and diagnosis of diseases. Much of the development in medical microwave imaging relates to breast imaging.

One approach to medical microwave imaging uses radar where reflections from the tissues are focused to create an image indicating locations where tissue properties change. Tomography is another approach where signals transmitted through the tissue are measured and compared to signals generated with a model; the properties of the model are iteratively updated until the measured and simulated signals converge. Other approaches to imaging with signals transmitted through tissues include tracking changes in amplitude, holography, scattered power mapping, and time-delay spectroscopy.

In all cases, there are challenges which involve: (i) coupling microwave signals into tissues, (ii) recording very low signal levels, and (iii) developing algorithms able to efficiently reconstruct an image, and interpreting results.

With respect to (i), coupling microwave signals into biological tissues challenges often result from the large mismatch between the electrical properties of the tissues and air.

With respect to (ii), recording very low-level signals-another limiting aspect of microwave imaging relates to the extremely low signals that may need to be detected. Those signals may be buried in other unwanted signals that need to be removed for proper imaging. Those unwanted signals may be reflections from the skin or reflections from other components in the imaging space. Complicated algorithms may be employed to remove these spurious responses, however, often do not effectively extract wanted signals. Fundamentally, extracting small signals from larger ones is a limiting factor as this requires microwave receivers to have significant dynamic range. The lack of ability to extract small signals inherently limits the sensitivity of the imaging or sensing system.

With respect to (iii), developing algorithms to efficiently reconstruct an image, and interpreting results—there are several limiting aspects related to the algorithms employed to create microwave images. The tomography approach involves estimating properties of a model in order to match simulated signals to measurements. The model is iteratively updated until the simulated and measured signals converge. This problem is inherently ill-conditioned, in part due to the multiple paths that signals travel through the tissue, and often converges to an incorrect solution. To avoid this scenario, additional information is typically added, such as ranges of properties values or information gathered from additional imaging modalities. This requires prior knowledge about the tissue, and further complicates the already complex imaging algorithms.

With radar approaches, the images track the location of changes in properties rather than measuring the actual properties. These changes may correspond to interfaces between healthy tissues, such as fat and glandular tissues, in addition to any lesions or anomalies present. Radar images contain multiple “hot spots” that require interpretation, typically in relation to the location of known lesions. Interpreting the amplitude of responses is particularly difficult, as several processing steps modify responses prior to image formation. Similar to tomography, radar images benefit from additional information about the tissue, such as ranges of properties, as this information improves focusing and detection.

With the majority of proposed microwave imaging approaches, a key complication is interpretation of data. As highlighted above, tomography involves complex algorithms that may not converge to the correct solution, while radar images typically contain multiple responses that require interpretation. To incorporate microwave imaging into the care path for women, it is important to relate these images to the clinical history of patients. Clinical imaging information typically consists of mammograms, with ultrasound and magnetic resonance imaging used to aid in diagnosis.

With most microwave scanners, the patient position is typically prone with the breast extending through a hole in the examination table. This position is substantially different from the readily available mammograms, complicating comparison between microwave images and clinically available imaging data. Ultrasound may be collected for local regions when investigating an anomaly, or with the patient in the supine position for automated breast ultrasound systems. Magnetic resonance imaging is used in sub-populations of patients (e.g. women at high risk of breast cancer or for tumour staging). MRI features the patient in the prone position, however does not use coupling liquids as is typical with microwave scans. The differences in the geometry of the breast during microwave imaging, mammography, ultrasound and MRI scans complicates interpretation of microwave breast images with clinically available information.

Other limitations in the prior art include perturbation of signals with other unwanted responses, low signal levels, and ability to image only the region of the breast close to the nipple. Many systems also provide only the locations of interfaces where properties change via reflection-based images, rather than mapping the properties themselves.

As such, there exists a need for alternative devices and methods to image biological tissues with microwave signals which may mitigate at least some of the above-noted challenges and limitations. The foregoing background is provided solely to facilitate an understanding of the art to which the present disclosure pertains, and is not an admission that any art is relevant prior art.

### SUMMARY OF THE INVENTION

In one aspect, disclosed are systems and methods of microwave scanning of biological tissue, which enable the measurement of biological tissue response to microwave signals without other unwanted signals, hence removing the need for suboptimal filtering techniques. The collected signals may be conditioned using techniques described herein.

In another aspect, disclosed are systems and methods of image reconstruction of biological tissues, which provide quantitative images related to the electrical properties of the tissues with a repeatable and stable technique.

In accordance with a broad aspect, there is provided a method for microwave scanning of biological tissue, comprising: activating antenna elements, in an antenna set, wherein the antenna set includes a transmitting antenna element in a transmitting antenna array, and one or more receiving antenna elements in a receiving antenna array; operating at least one microwave transmitter to generate an interrogation microwave signal for transmission, via the transmitting antenna element, into a biological tissue; receiving, via at least one microwave receiver, one or more received microwave signals from each of the one or more receiving antenna elements; applying pre-conditioning to each of the received microwave signals to generate corresponding conditioned microwave signals, wherein the pre-conditioning isolates tissue response properties from extraneous response factors; analyzing the conditioned microwave signals to determine one or more tissue response properties, associated with the biological tissue; and generating an output based on the determined one or more tissue response properties.

In some examples, activating the antenna elements comprises operating a first switching network to couple the transmitting element to the at least one microwave transmitter, and operating a second switching network to couple the one or more receiving elements to the at least one microwave receiver.

In some examples, activating the antenna elements comprises operating the microwave transmitter coupled to the transmitting antenna element, and coupling a microwave receiver coupled to the receiving antenna element.

In some examples, the biological tissue is positioned in a scanning region between the transmitting and receiving antenna arrays, and contacts the antenna arrays.

In some examples, a dielectric cover material is coupled to one or more of transmitting and receiving antenna arrays, and contacts the biological tissue within the scanning region.

In some examples, the dielectric covering material has a relative permittivity below 5 and a maximum thickness of 2 mm, and more preferably, a relative permittivity around 2.5 and thickness below 0.5 mm.

In some examples, applying the pre-conditioning to a microwave signal comprises: identifying a transmitting and receiving antenna pair, associated with the microwave signal; determining one or more configuration parameters of the antenna arrays; based on the determining, applying path-specific correction factors to the microwave signal to generate a corrected microwave signal; and applying antenna response compensation factors to the corrected microwave signal to generate a conditioned microwave signal.

In some examples, the path-specific correction factors include correction factors for phase and magnitude correction for each the receiving pathway and transmission pathway associated with the transmitting and receiving antenna pair.

In some examples, the configuration parameters of antenna arrays correspond to one or more: (i) the positional spacing of antenna elements on each array; (ii) a spatial axial distance between the antenna arrays and/or (iii) a rotational orientation of the antenna.

In some examples, the antenna response compensation factors include antenna gain compensation factor and phase center compensation factors, and the antenna compensation factors are associated with: (i) the biological tissue type, and (ii) the configuration parameters of antenna arrays.

In some examples, the antenna response compensation factors and path-specific correction factors, are generated by simulation tools, which generate reference compensation and correction factors.

In some examples, each antenna array comprises a plurality of slot antenna elements, each slot antenna including a respective shielding interface.

In some examples, each switching network comprises one or more switching subnetworks, and each antenna array comprises one or more antenna subarrays.

In accordance with another broad aspect, there is provided a system for microwave scanning of biological tissue, comprising: a transmitting and a receiving antenna array, each antenna array comprising a plurality of antenna elements, wherein the antenna arrays are separated along an axis, by an axial separation distance, to define a scanning region for receiving biological tissue; a first and a second switching network, each coupled to a respective transmitting and receiving antenna array, wherein the switching network comprises a plurality of switching elements; at least one microwave transmitter coupled to the transmitting antenna array, via the first switching network; at least one microwave receiver coupled to the receiving antenna array, via the second switching network; and a controller coupled to the first and second switching network, and the at least one microwave transmitter and receiver, and operable to perform the methods of any of the preceding paragraphs.

In accordance with another broad aspect, there is provided a system for microwave scanning of biological tissue, comprising: a transmitting and a receiving antenna array, each antenna array comprising a plurality of antenna elements, wherein the antenna arrays are separated along an axis, by an axial separation distance, to define a scanning region for receiving biological tissue; at least one microwave transmitter coupled to the transmitting antenna array; at least one microwave receiver coupled to the receiving antenna array; and a controller coupled the at least one microwave transmitter and receiver, and operable to perform the methods of any one of the preceding paragraphs.

In some examples, the at least one microwave transmitter comprises a plurality of microwave transmitters, each microwave transmitter coupled to a separate antenna element of the transmitting antenna array, and the at least one microwave receiver comprises a plurality of microwave receivers, each microwave receiver coupled to a separate antenna element of the receiving antenna array.

In accordance with another broad aspect, there is provided an antenna assembly for use with a system for microwave scanning of biological tissue, comprising: at least two antenna arrays, wherein the antenna arrays are separated along an axis, by an axial separation distance, to define a scanning region for receiving biological tissue; and a dielectric covering material coupled to and overlaying each antenna array, wherein the dielectric covering material contacts the biological tissue inserted in the imaging region defined.

In some examples, each antenna array comprises a plurality of slot antennas.

In some examples, each slot antenna includes a respective shielding interface.

In some examples, the dielectric covering has a relative permittivity below 5 and a maximum thickness of 2 mm, and more preferably, a relative permittivity around 2.5 and thickness below 0.5 mm.

### BRIEF DESCRIPTION OF THE DRAWINGS

So that the manner in which the above recited features of the present invention can be understood in detail, some embodiments are illustrated in the appended drawings. It is to be noted, however, that the appended drawings illustrate only typical embodiments of this invention and are therefore not to be considered limiting of its scope.

FIG. 1 is a perspective view of an example device for microwave imaging, in accordance with disclosed examples.

FIG. 2A is an example two-dimensional (2D) image reconstruction of electrical permittivity properties of an example breast tissue.

FIG. 2B is an example three-dimensional (3D) image reconstruction of an example breast tissue.

FIG. 3A shows isometric views of two curved antenna arrays and a mechanical system, according to some embodiments.

FIG. 3B shows isometric views of an angled antenna array and a mechanical system, according to some embodiments.

FIG. 3C is an isometric view of an example antenna array, according to some embodiments.

FIG. 4A is an example microwave imaging system, in accordance with disclosed examples.

FIG. 4B is another example microwave imaging system, in accordance with disclosed examples.

FIG. 5A is an illustration of two antenna arrays, and a number of signal paths extending between the antenna arrays.

FIG. 5B is a two-dimensional view of two antenna arrays, and a plurality of signal paths extending between the antenna arrays.

FIG. 6 illustrates an isometric view of an antenna array that includes a plurality of slot antennas integrated in a main printed circuit board (PCB).

FIG. 7 illustrates another example antenna array that includes a plurality of slot antennas mounted on a main printed circuit board (PCB).

FIG. 8A illustrates an example slot antenna in accordance with the teachings herein, as well as an exploded view of the slot antenna.

FIG. 8B shows various example views of the slot antenna of FIG. 8A, including a top plan view (top left), a perspective view (top right), a cross-sectional elevation view along the section line A-A′ in the top left image (bottom left) and a bottom perspective view (bottom right).

FIG. 8C shows another example slot antenna in accordance with the teachings herein.

FIG. 8D is an isometric view of the slot antenna integrated in a multilayer printed circuit board according to some embodiments.

FIG. 8E is an isometric view of the slot antenna and its microstrip feed according to some embodiments.

FIG. 8F shows top views of various possible slot shapes of the slot antenna according to some embodiments.

FIG. 9 is a plot showing the coupling between antenna elements, with and without shielding.

FIG. 10A shows a ridged waveguide on top of the slot antenna according to some embodiments.

FIG. 10B is a partially transparent view, of the waveguide of FIG. 10A.

FIG. 10C shows various views of the waveguide of FIG. 10A, including a front elevation view (top left), a side elevation view (top right) and a bottom-up view (bottom left).

FIG. 11 is a side view of a slot antenna and a thin dielectric cover to separate the skin from the radiating component, according to some embodiments.

FIG. 12A is an example plot showing the effect of applying a thin low permittivity dielectric cover to antenna arrays.

FIG. 12B is another example plot showing the effects of applying a dielectric cover to antenna arrays.

FIG. 13A is an example method for scanning biological tissue using microwave signals.

FIG. 13B is an example method for microwave signal pre-conditioning.

FIG. 13C is another example method for microwave signal pre-conditioning.

FIG. 14A shows an example nearfield radiation pattern in tissues that are predominantly fatty.

FIG. 14B shows an example nearfield radiation pattern in tissues with greater glandular content.

FIG. 15A shows an example nearfield phase pattern in tissues that are predominantly fatty.

FIG. 15B shows an example nearfield phase pattern in tissues with greater glandular content.

FIG. 16A is a plot of gain compensation for two different tissue types, and plotted for different frequencies.

FIG. 16B is a plot of phase center compensation for two different tissue types, and plotted for different frequencies.

FIG. 17A is a plot showing a resulting correction using gain compensation on the attenuation of the microwave signal.

FIG. 17B is a plot showing a resulting correction using phase centre compensation on the phase of the microwave signal.

FIGS. 18A and 18B show plots of the results of correction for relative permittivity (FIG. 18A) and conductivity (FIG. 18B) using signals when antenna response compensation is applied compared to the original signal, material properties are calculated with a Nicolson Ross technique.

FIG. 19A illustrates an example simulation model.

FIG. 19B is a plot comparing transmission coefficients versus frequency when transmitting microwave signals through canola oil (fat mimicking liquid), and showing measured versus simulated results.

FIG. 19C is a plot comparing transmission coefficients versus time when transmitting microwave signals through canola oil (fat mimicking liquid), and showing measured versus simulated results.

FIG. 19D is a histogram of the calculated average permittivity of a tissue using 333 simulated signals transmitted through the biological tissue model Group 1 Low.

FIG. 19E is a histogram of the calculated average permittivity of a tissue when applying the antenna response compensation to the 333 simulated transmission signals going through the biological tissue model Group 1 Low.

FIG. 19F is a histogram of the calculated average permittivity of a bag filled with glycerin using approximately 300 measured transmission signals.

FIG. 19G is a histogram of the calculated average permittivity of a bag filled with glycerin when applying the antenna response compensation to the approximately 300 measured transmission signals. The antenna response compensation factors for group 3 Med are used.

FIG. 19H is a histogram of the calculated average permittivity of a bag filled with water using approximately 300 measured transmission signals.

FIG. 19I is a histogram of the calculated average permittivity of a bag filled with water when applying the Group 1 Med antenna response compensation to the approximately 300 measured transmission signals.

FIG. 19J is a histogram of the calculated average permittivity of a bag filled with water when applying the Group 3 Med antenna response compensation to the approximately 300 measured transmission signals.

FIG. 20 is a simplified hardware block diagram for an example controller.

### DESCRIPTION OF THE INVENTION

Disclosed embodiments relate to a method and system for microwave scanning of biological tissue.

### I. Definitions

The present invention relates generally to methods and systems for imaging biological tissues using microwave signals, and preferably providing quantitative images of the electrical properties of biological tissues at microwave frequencies. Any term or expression not expressly defined herein shall have its commonly accepted definition understood by a person skilled in the art.

“Biological tissue” refers to any tissue of an organism, including animal or human tissues. The biological tissues may be any limbs, head, neck or torso, and in a particular non-limiting example, the biological tissue is human breast tissue.

“Memory” refers to a non-transitory tangible computer-readable medium for storing information in a format readable by a processor, and/or instructions readable by a processor to implement an algorithm. The term “memory” includes a plurality of physically discrete, operatively connected devices despite use of the term in the singular. Non-limiting types of memory include solid-state, optical, and magnetic computer readable media. Memory may be non-volatile or volatile. Instructions stored by a memory may be based on a plurality of programming languages known in the art, with non-limiting examples including the C, C++, Python™, MATLAB™, and Java™ programming languages.

“Processor” refers to one or more electronic devices that is/are capable of reading and executing instructions stored on a memory to perform operations on data, which may be stored on a memory or provided in a data signal. The term “processor” includes a plurality of physically discrete, operatively connected devices despite use of the term in the singular. Non-limiting examples of processors include devices referred to as microprocessors, microcontrollers, central processing units (CPU), and digital signal processors. In some embodiments, the processing unit comprises a stand-alone embedded processor system, optionally connected to a standard computer. In some embodiments, the embedded processor system may comprise a microcontroller and a Field Programmable Gate Array (FPGA). The processor is linked to a memory which includes instructions to implement the scanning and imaging steps described herein.

“Microwave” frequencies refers to electromagnetic (EM) waves in the microwave region, generally considered to be in the range of about 300 MHz to about 300 GHz, corresponding to wavelengths in the range of between about 1 m and 1 mm.

### II. General Overview

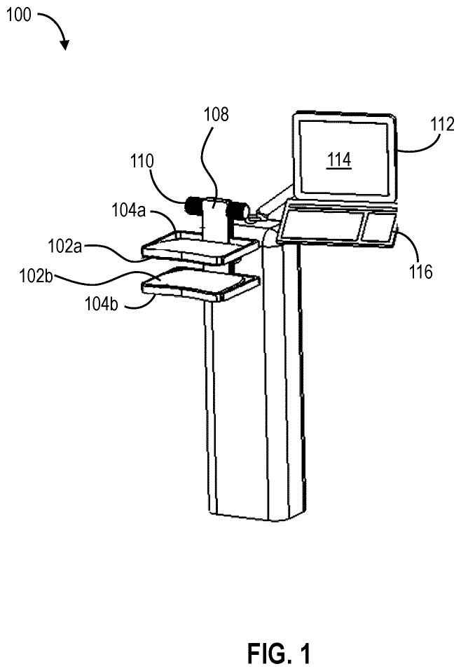

FIG. 1 illustrates an example device (100) for microwave imaging of biological tissue (e.g., breast tissue), in accordance with disclosed embodiments. As explained herein, device (100) can use microwave signals to scan tissue, and determine various properties of that tissue.

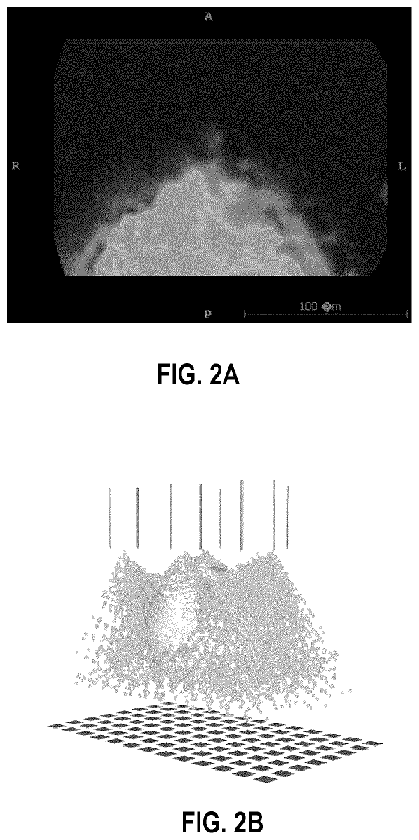

To this end, FIGS. 2A and 2B exemplify outputs generatable by the device (100). These include two-dimensional (2D) image outputs of biological tissue (FIG. 2A) and/or three-dimensional (3D) image outputs (FIG. 2B). In other examples, various other data outputs can be generated, including numerical scores, etc.

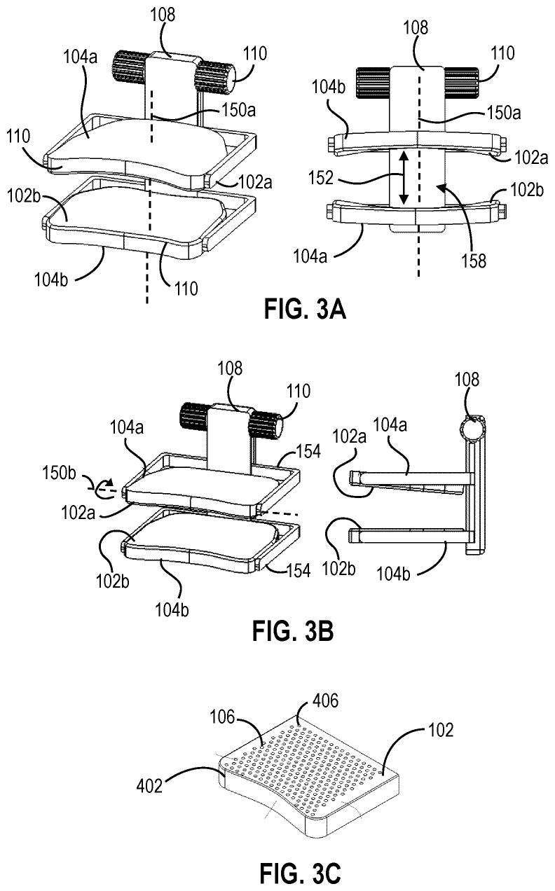

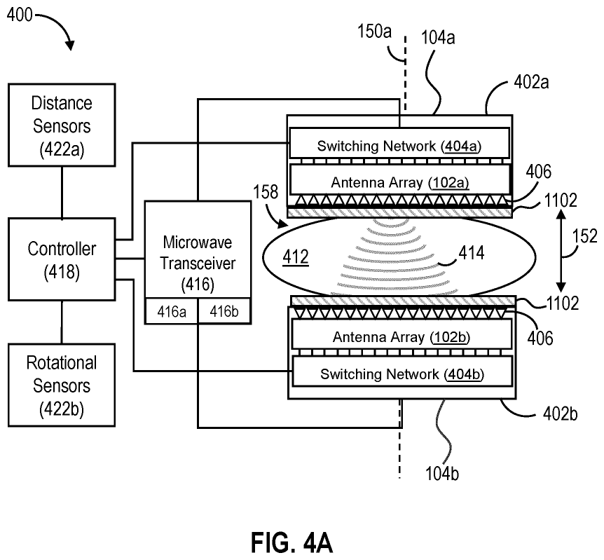

As shown in FIG. 1, device (100) includes at least two opposing antenna arrays (102a), (102b). The antenna arrays (102a), (102b) may be aligned along a common orientation axis (150a) (FIG. 3A), and oriented such that the antenna arrays (102a), (102b) are directed to face each other and otherwise spaced apart by a spatial axial distance.

As used herein, the “spatial axial distance” (also referred to herein throughout interchangeably as “spatial distance” or “axial distance” or “separation distance”) between the antenna arrays (102a), (102b), refers to the separation axial distance (152) (FIG. 3A), defined along the orientation axis (150a), between the spaced antenna arrays (102a), (102b). A gap (or void) (158) is defined by the separation distance (152). In some examples, the gap (158) is also referred to herein as the scanning region (158) (or the “tissue scanning region”). This is because a biological tissue requiring scanning, is insertable within the region (158) and between the two antenna arrays (102).

As detailed herein, in some examples, it is possible that each antenna array (102a), (102b) is segmented into two or more sub-antenna arrays. In these cases, more than two antenna arrays are provided, with the multiple antenna arrays also being aligned to face one another. Further, the antenna arrays (102a), (102b) are not necessarily disposed vertically over each other. For instance, they may be disposed laterally, or at any other desired angle with respect to each other.

In an example application where the device (100) is used to scan breast tissue, a patient may be in a sitting or standing position, and may position their breast tissue between the opposing antenna arrays (102a), (102b), and within the scanning region (158). In other examples, other types of biological tissue can be positioned between the opposing antenna arrays (102a), (102b).

As best shown in FIGS. 3A to 3C, each antenna array (102a), (102b) may be housed in a separate corresponding housing (104a), (104b). Each antenna array housing (104a), (104b) can include a respective major antenna surface (106) (FIG. 3C), which comprises the respective antenna array (102a), (102b), and which is configured to contact the scanned tissue.

The major antenna surfaces (106) may have any desirable shape or configuration. In some examples, the major surfaces (106) are substantially horizontal (FIG. 3C). Otherwise, as shown in FIG. 3A, the surfaces (106) are contoured or curved (e.g., concavely curve). In this manner, the antenna array (102), itself, is also contoured or curved. In at least one example, the contouring of the major surface (106) (and thereby, the antenna array (102)), allows the array to better fit around certain types of body tissue (e.g., breast tissue).

Additionally, or in the alternative, the housing front edge surface (110) may also be curved (FIG. 3A). This also allows the housing to better accommodate specific biological tissues or body parts, including but not limited to the breast, the chest, the rib cage, or the head. For example, when imaging a human breast, it may be desirable for the front edge (110) of the antenna array housing to fit the curvature of the rib cage for better comfort.

In at least one example, the antenna array housing (104) is covered with a microwave-transparent cushioning (or deformable material) to improve the comfort of the device. For example, the cushioning may include foam or gel pads.

As noted above, it is possible for the antenna arrays (102a), (102b) to be split into a plurality of antenna subarrays. In these cases, the housings (104) may also be segmented into sub-housings, each accommodating a respective antenna subarray. This may permit, for instance, a better fit of the shape of the biological tissues to be scanned. For example, four housings may be used at different adjustable angles to surround different shapes such as a human breast, neck or a head.

As exemplified in FIG. 3B, the antenna array housings (104a), (104b) (and therefore, the antenna arrays (102)) can be pivotable. For instance, the housings (104a), (104b) can each pivot around a rotation axis (150b). Rotation axis (150b) can be orthogonal to the orientation axis (150a). In this example, each housing (104) is pivotably mounted, to a mounting arm (154). The pivoting allows the antenna arrays (102a), (102b) to be angled front to back.

In some examples, the angle at which the antenna arrays (102) are set may be pre-defined such that the device can be locked into specific angles. In case of imaging of the breast, these positions may correspond to craniocaudal (CC) and mediolateral-oblique (MLO) views.

The antenna arrays (102a), (102b) can also be angled to better fit the shape of the biological tissues to be scanned and/or to increase the diversity of data recorded. For example, the anterior-posterior angle and/or the lateral angle between the antenna arrays (102a), (102b) may be adaptable to the biological tissues to scan.

Referring back to FIG. 1, device (100) can include a mechanical system (108). Mechanical system (108) can be used to support and position the antenna housings (104a), (104b). A knob (110) can allow adjusting the mechanical system (108), such as to adjust the spatial axial distance (152), between the opposing arrays (102a), (102b).

More particularly, the mechanical system (108) may retain the two or more antenna arrays (102) oriented in opposed fashion, such that fields emitted by the antenna arrays (102a), (102b) are directed towards the opposing array. The mechanical system (108) provides the ability to adjust the separation distance (152) (FIG. 3A) between antenna arrays (102a), (102b) to allow inserting biological tissues, to be scanned, within the scanning region (158). The separation distance (152) can be adjusted and reduced, via mechanical system (108), such that the antenna arrays (102a), (102b) contact the biological tissues to be scanned.

In at least one example, the mechanical system (108) provides a manual or power actuated adjustment input to manually change the separation distance (152) between antenna arrays (102a), (102b). The mechanical system (108) may include position indicators, such as a magnetic reader and strip, or other means which provide the ability to measure the separation distance (152) between arrays (102a), (102d), and/or measure the position of an antenna array relative to other arrays.

Mechanical system (108) can also control rotation about rotation axis (150b), such that the at least two antenna arrays (102a), (102b) (FIG. 3B) are at an angled position to provide a different view of the biological tissues, as explained above. The mechanical system (108) may provide a way to measure the angle at which the antenna arrays (102a), (102b) are located.

In some examples, it is also possible that the mechanical system (108) allows only one antenna array (102) to be angled while the other antenna array (102) remains essentially fixed. For example, this feature can be used where there are upper and lower antenna arrays, the upper one may be adjustable while the lower one is fixed.

The antenna arrays (102) may also have the ability to translate laterally (the array assembly) so that microwave measurements can be recorded with additional antenna element locations, hence effectively increasing the density of measurement. For example, if one of the two arrays is moved laterally by a distance equal to one-half the lateral distance between two adjacent antennas, then readings taken from one position can be combined with readings from the other position to effectively improve the resolution of that array. In some examples, an actuation mechanism (not shown) is provided to move antennas laterally.

As shown in FIG. 1, device (100) can also include a computer terminal (112). Computer terminal (112) can include a computer display screen (114) and an input interface (116) (e.g., keyboard and/or trackpad or mouse). The computer terminal (112) can allow the operator to control the device.

As provided herein, a measurement protocol may be provided to guide the use of the device (110) in scanning (e.g., imaging) biological tissue.

### III. System for Microwave Scanning of Biological Tissue

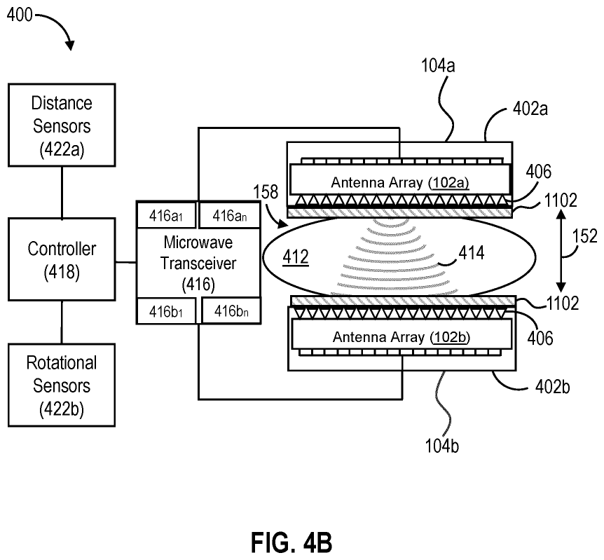

FIG. 4A illustrates an example microwave scanning system (400), in accordance with disclosed embodiments. In some examples, microwave scanning system (400) is incorporated into the microwave imaging device (100) (FIG. 1).

As shown, microwave scanning system (400) includes at least two antenna assemblies (402a), (402b). Each antenna assembly (402) may be disposed within a respective antenna housing (104a), (104b).

Each antenna assembly (402) includes a corresponding antenna array (102a), (102b) coupled to a corresponding switching network (404a), (404b). Each antenna array (102a), (102b) includes a plurality of antenna elements (406) (e.g., two or more antenna elements), which are coupled to the respective switching network (404a), (404b). In operation, each antenna array (102a), (102b) is operated to transmit (e.g., emit) and/or receive microwave signals (414). In some examples, as discussed herein, the system (400) may not necessarily include the switching networks (404) (FIG. 4B).

As noted above, it is possible that one, or both, of the antenna assemblies (402) includes antenna arrays (102a), (102b) that are sub-divided (e.g., segmented) into two or more subarrays. In turn, the antenna subarrays may be coupled to the same corresponding switching network (404). In other examples, one or more of the switching networks (404) are also segmented into two or more subnetworks. The subnetworks may be coupled to the same or different antenna arrays and/or antenna subarrays.

Antenna arrays (102a), (102b) are disposed in an opposing configuration along orientation axis (150a), and are spaced apart to define the scanning region (158) extending along the separation distance (152). As discussed, scanning region (158) can receive a biological tissue (412) (or any portion thereof) to be scanned, e.g., a human breast tissue. In this manner, the opposing antenna arrays (102a), (102b) are placed axially on either side of the target tissue (412) to be scanned.

In some examples, antenna housings (104a), (104b) are adapted to be in contact with the target tissue (412). For example, mechanical system (108) can be adjusted such that the spatial distance (152) is adjusted to accommodate different sizes of biological tissue, and bring the antenna housings (104)—and therefore, the antenna arrays (102)—into contact with the scanned tissue (412).

System (400) also includes a microwave transceiver (416) coupled to the antenna arrays (406), via the switching networks (404). A controller (418) is further provided, which is coupled to both the microwave transceiver (416) and the switching networks (404).

Each of the microwave transceiver (416), switching networks (404) and controller (418) are now discussed herein, in greater detail.

### (i.) Microwave Transceiver.

In at least one example, microwave transceiver (416) includes: (i) at least one microwave source transmitter (416a) to generate transmitted (e.g., emitted) microwave signals (414), and, (ii) at least one microwave receiver (416b) to receive coherently the microwave signals (414).

In some examples, it is possible that the microwave transmitter (416a) and receiver (416b) are provided as separate units, rather than being integrated into a single transceiver, as shown.

Microwave transceiver (416) can also include a plurality of transmitters (416a) and/or receivers (416b). For instance, microwave transceiver (416) can include multiple transmitters (416a) working at different frequencies, that are to be able to send signals in parallel. Similarly, multiple receivers (416b) may be provided to receive signals simultaneously in parallel. In these examples, the switching networks (404a), (404b) may not be necessarily included.

For example, in FIG. 4B, a plurality of microwave transmitters (416a₁)-(416aₙ) are included, each connected to a separate antenna element (406), in the transmitting antenna array (102a). Further, a plurality of microwave receivers (416b₁)-(416bₙ) are included, each connected to a separate antenna element (406), in the receiving antenna array (102b).

Accordingly, in at least one example, there are an equal number of microwave transmitters (416a) as transmitting antenna elements (406), in transmitting antenna array (102a). Further, there are equal number of microwave receivers (416b) as receiving antenna elements (406), in the receiving antenna array (102b). At least the transmitters (416a) may be operating at the same and/or different frequencies.

Although FIG. 4B exemplifies, for ease of illustration, a single connection line between the microwave transmitters and receivers (416a), (416b), it is understood that the connection line in-fact comprises a plurality of connection lines, coupling respective microwave transmitters (416a₁)-(416aₙ) and microwave receivers (416b₁)-(416bₙ), with respective individual antenna elements (406) in the respective transmitting and receiving antenna arrays (102a), (102b).

In at least one example, it is possible that a switching network (404) is provided for one antenna array, and not the other. It is also possible to provide multiple transmitters and receivers (416a), (416b), which are coupled to the same or different switching networks (or switching subnetworks). For instance, if there are less microwave transmitters or receivers (416a), (416b) than corresponding antenna elements (406), then some of these microwave transmitters or receivers (416a), (416b) may be coupled to switching subnetworks (414), while others are coupled directly to corresponding antenna elements (406).

In at least one example, microwave transmitter(s) (416a) are operable to generate microwaves in a frequency range between 100 MHz to 10.6 GHz, and in some examples, between 2 GHz to 8 GHz. Suitable microwave transceivers (416) are well known in the art.

### (ii.) Switching Network.

Switching networks (404a), (404b) allow for coupling any antenna element (406), in the corresponding antenna array (102a), (102b), to either the microwave transmitter (416a) or microwave receiver (416b), which may be incorporated into a transceiver (416). Accordingly, the switching networks (404a), (404b) are used for selecting transmitting and receiving antenna elements (406).

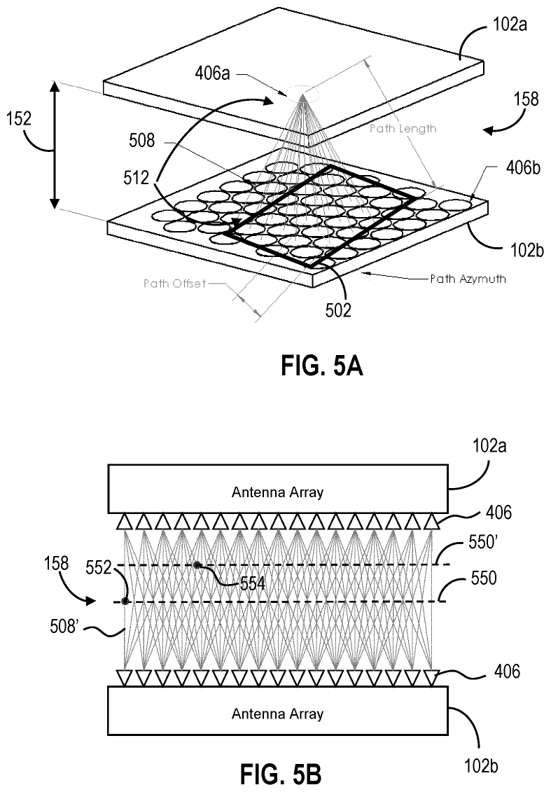

By way of example, as shown in FIG. 5, antenna array (102a) can be designated as the transmitting antenna array, while antenna array (102b) is designated as the receiving array. In this case, switching network (404a) may couple antenna element (406a)—in transmitting array (102a)—to the microwave transmitter (416a). Further, switching network (404b) may individually couple each of a group (502) of antenna elements (406b)—in the receiving array (102a)—to the microwave receiver (416b). Accordingly, the antenna element (406a) is used to emit (e.g., transmit) a microwave signal, while the group of antenna elements (502) are used for receiving the microwave signal.

As used here, an ‘activated’ antenna element (406), is an antenna element (406) coupled, e.g., by the switching network (404), to either the microwave transmitter (416a) or receiver (416b), e.g., in the microwave transceiver (416). In the case of FIG. 4B, an ‘activated’ antenna element is an antenna element that is directly coupled to a transmitter or receiver that is being operated (e.g., turned-on) by the controller (418). More generally, an activated antenna element is an antenna element that is being actively used to transmit/receive signals.

To this end, a signal path (508) is defined between each pair of activated transmitting and receiving antennas (FIG. 5A). That is, each signal path (508) is associated with a respective transmitting and receiving antenna element, located on opposing antenna arrays. As detailed herein, an appreciated advantage of the design of the antenna arrays (102a), (102b)—in combination with the switching networks (406)—is to enable the selection of a large plurality of different signal paths.

In at least one example, if one antenna array (102) has 250 antennas (x) and another array has 250 antennas (y), then there are potentially x*y (e.g. 62,500) different signal paths available between different antenna pairs (see e.g., FIG. 5B). The switching networks (404a), (404b) are therefore operated to connect some or all of the available signal paths (508). In some examples, the scanning system (400) allows for the selection of between about 1,500 to about 20,000 signal paths.

As explained herein, the provision of a greater number of signal paths (508), enhances the scanning resolution of the system (400). For example, different signal paths (508) are generated to intersect through different cross-sectional portions (e.g., areas) of the biological tissue located within the scanning region (158). The greater number of signal paths allows for acquiring a greater volume of data regarding tissue response.

In at least one example, the switching networks (404) each comprise a plurality of microwave switches, as known in the art, and connected to each antenna element (406) in the respective antenna array. The microwave switches are controllable, e.g., via controller (418), to selectably couple specific antenna elements (406) to either the microwave transmitter (416a) or receiver (416b). In some examples, the microwave switches are solid-state microwave switches, which are integrated in the antenna array printed circuit board (PCB).

Switching networks (404a), (404b) may also incorporate multiple microwave transceiver integrated circuits located on the same PCB. In this case, a transceiver is connected to each antenna, and the transceiver itself can be switched between transmitting and receiving modes.

### (iii.) Controller.

Controller (418) (FIG. 4A) can perform various functions including controlling the microwave transceiver (416) (e.g., transmitter (416a) and/or receiver (416b)) and controlling the switching networks (404a), (404b). In some examples, controller (418) also provides some signal processing and analysis functionality, as disclosed herein.

More particularly, controller (418) operates the switching networks (404a), (404b) to select a plurality of pairs of antenna elements (406)—in each array (102a), (102b)—to connect to the microwave transmitter (416a) and/or receiver (416b), wherein each transmitting antenna element (406) may be separately paired with each of a plurality of receiving antennas (406).

In other examples, where switching networks (404a), (404b) are not necessarily provided (FIG. 4B), then controller (418) may be operable to control the correct microwave transmitters (416a₁)-(416aₙ) and microwave receivers (416b₁)-(416bₙ), which are coupled to the corresponding transmitting and receiving antenna elements (406), in each array (102a), (102b), that are desired to be activated.

To this end, while for ease of explanation, the remaining discussion herein assumes that switching networks (404) are provided in system (400), it will be understood that all described examples can be identically replicated using the system (400) in FIG. 4B, e.g., by controlling one of a number of microwave transmitters and/or receivers (416a), (416b), as opposed to the switching networks, to activate different antenna elements.

The controller (418) also operates the microwave transceiver (416) to generate and receive microwave signals, via activated antenna elements. Controller (418) can also process the received microwave signal to determine one or more tissue response properties (e.g., electrical properties), associated with the scanned biological tissue.

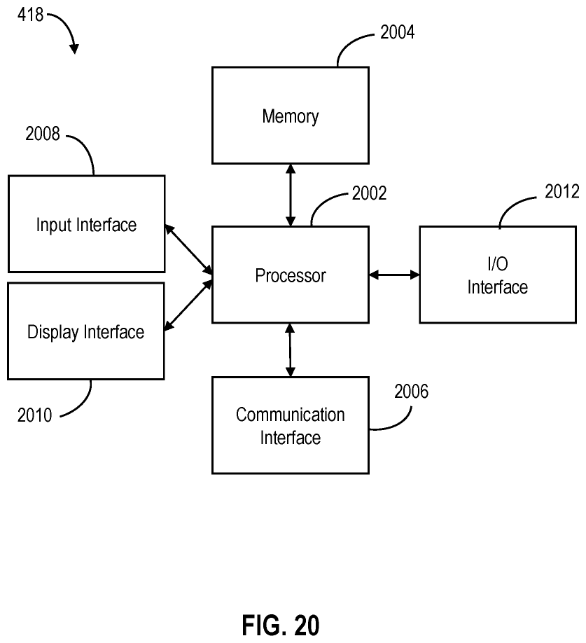

As provided herein, with reference to FIG. 20, controller (418) can include at least one processor (2002), which is coupled to a memory (2004). In some examples, processor (2002) is also coupled to one or more of a communication interface (2006), an input interface (2008), an output interface (2010) and an I/O interface (2012).

Controller (418) may comprise, or otherwise may by incorporated within, computer terminal (112) (FIG. 1). In other examples, controller (418) is separately provided, and may be coupled to computer terminal (112).

### IV. Antenna Array

The following is a more detailed discussion of a design configuration for an antenna array (102), which may be used by itself or in combination with any of the features disclosed herein, e.g., including microwave imaging system (400).

### (i.) Configuration of Antenna Elements in Antenna Array.

As noted above, each antenna array (102) comprises a plurality of individual antenna elements (406) (FIG. 4A), dispersed on a primary surface (106) of a housing (104).

As provided herein throughout, a unique aspect of the scanning system (400) is the provision of a plurality of antenna elements (406) in each opposing antenna array (102). This allows controlling the switching networks (404) (FIG. 4A) (or the microwave transmitters/receivers in FIG. 4B) to alternate between many signal paths (508) associated with different pairs of transmitting and receiving antenna elements. In turn, this provides for higher resolution scanning of the biological tissue.

Further, the disclosed antenna assemblies can scan a large volume of tissue while maintaining the antenna arrays (102) in a fixed relative orientation and/or position. This is because the antenna arrays (102) are capable of generating a multiple of signal paths (508)—travelling between different pairs of antennas (406), and at different angles—whereby such signal paths (508) intersect and travel through different parts of the biological tissue. This avoids the necessity of having antenna assemblies that require movement or translation mechanisms to scan different regions of the biological tissue, and therefore increase the scanning time. In some examples, the antenna array (102) includes dozens or hundreds of antenna elements (406) to allow for a large number of signal paths. In at least one example, the disclosed embodiments can scan large volumes of tissue in as little as 30 seconds of time.

To this end, the antenna elements (406) can be distributed around a given antenna array (102) in any desired pattern configuration (e.g., FIG. 3C). By way of non-limiting examples, some or all of the antenna elements (406) can be distributed in patterns including a triangular lattice (e.g., offset rows), square lattice (e.g., aligned rows and columns), rectangular lattice (e.g., aligned rows and columns), or a hexagonal lattice. Alternatively, or in addition, the antenna array (102) may have some or all of the antennas (406) distributed randomly.

In at least one example, some or all of the antenna elements (406), in each antenna array (102), are slot antennas.

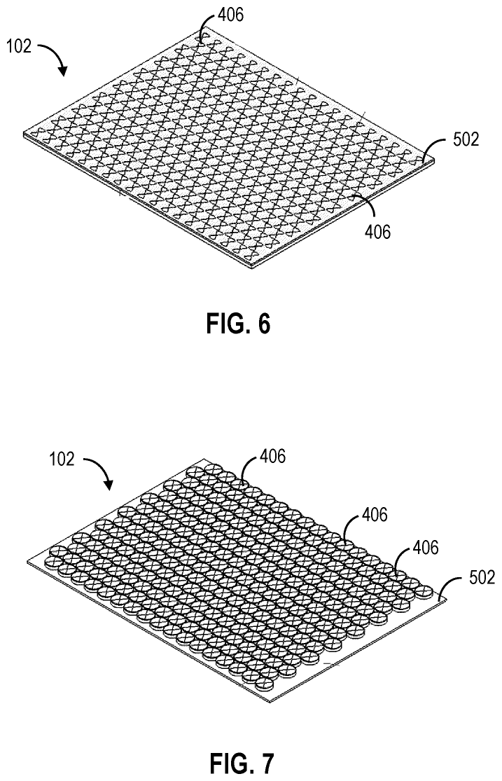

As best shown in FIGS. 6 and 7, an appreciated advantage of using a slot antenna design is that slot antennas have a compact design that facilitate mounting many (e.g., hundreds or thousands) of antennas in close proximity and over small cross-sectional surface areas, e.g., on a PCB (502). In at least one example, the slot antenna design allows for 1,680 slot antennas on a surface area of 30 cm×24 cm. This design configuration is particularly suited for the disclosed application, as it facilitates assembling antenna arrays (102) with large numbers of antennas (406), that are controllable via the switching network (404) (or via a plurality of microwave transmitters and receivers, as in FIG. 4B), to generate many combinations of signal paths (508) during tissue scanning (FIGS. 5A-5B) , thereby acquiring higher resolution tissue property response data from different parts of the scanned biological tissue. As further explained below, slot antennas are also less susceptible to the presence of biological tissue in close proximity.

FIG. 6 exemplifies an antenna array (102) where the slot antennas (406) are integrated directly into the mounting substrate (502) (e.g., PCB (502)) during fabrication. FIG. 7 exemplifies an antenna array (102), where the slot antennas (406) are coupled over (e.g., mounted over, or soldered onto) the mounting substrate (502) (e.g., PCB (502)).

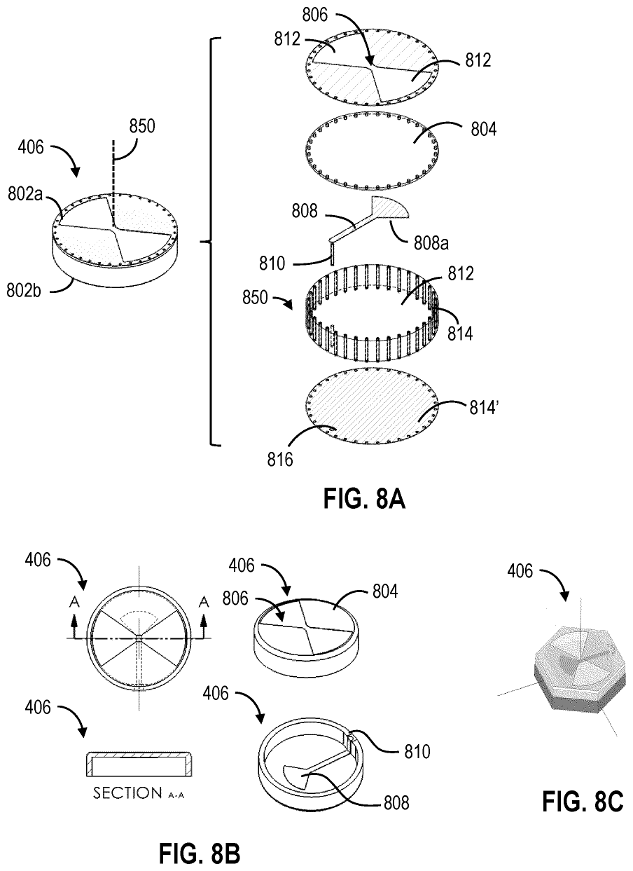

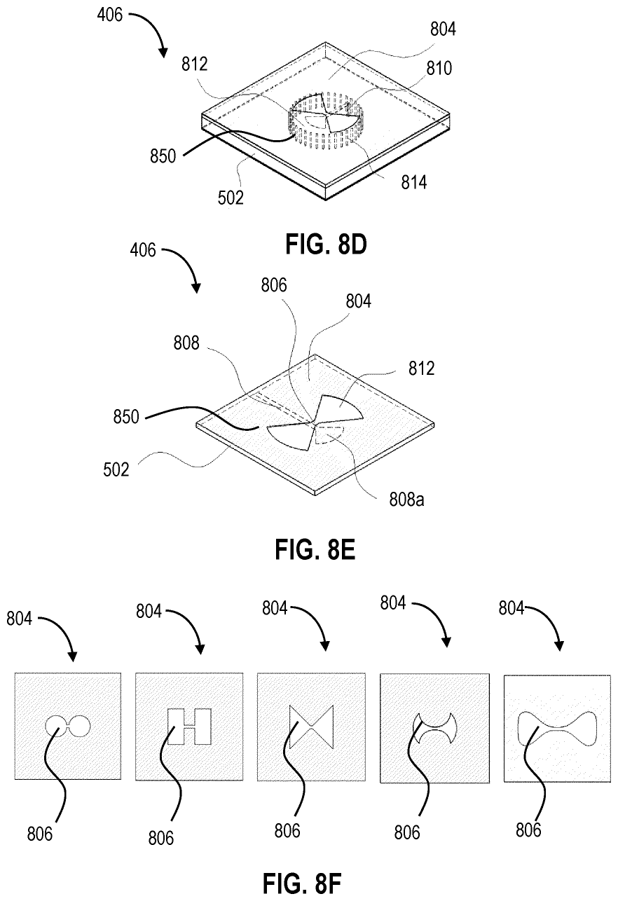

While any slot antenna design known in the art may be used, FIGS. 8A-8B exemplify a design for a slot antenna (406) used in disclosed examples. The examples in FIGS. 8A and 8B can be used with the antenna array in FIG. 7, e.g., the slot antennas (406) coupled to the mounting substrate (502).

As shown, in the upright position, the exemplified slot antenna (406) can extend between an upper side (802a) and a lower side (802b), along axis (850). While relative terms such as “upper” and “lower” are used herein throughout, the disclosed slot antennas are not limited to any particular orientation.

The upper side (802a), of each slot antenna, may be exposed for transmitting and/or receiving microwave signals, while the lower side (802b) may be mounted to a substrate (e.g., a PCB).

More generally, the slot antenna (406) can include a metallized substrate (804), on which part of the metallization is removed to form a slot (806) (although, for ease of illustration, the slot (806) is shown separately in FIG. 8A from the metallized substrate (804) on which it is formed). In the assembled state, the metallized substrate (804) forms the upper side (802a) of the slot antenna.

Slot (806) may be fed on the other side of the metallized substrate (804). A conductive feeding strip (808) (e.g., microstrip line or strip line), oriented substantially perpendicular to the slot (806), may be located directly below the substrate (804), and otherwise coupled to the substrate (804). The feeding strip ends past the slot (806), forming a stub (808a). Many stub shapes can be used for both the microstrip feed and the slot. For example, radial stubs can be used to optimize the space used by the antenna element.

In some examples, the slot antennas (406) are mounted (e.g., manufactured or fabricated) on a thin permittivity substrate, such as standard printed circuit board (PCB) laminates, engineered plastic or ceramic materials.

As best shown in FIGS. 8A and 8D, the feeding strip (808) may be connected to a further layer in a PCB stack up (502) through a small, metallized hole (via) (810) (FIGS. 8A and 8D). In this case, the thickness of the substrate below the microstrip may be electrically thicker (in terms of wavelength) than the substrate used for the slot.

In at least one example, the via (810) (FIG. 8A) connecting the feeding strip (808) may extend to the top metallized substrate layer (804) (where the slot is located). In these examples, the via (810) can be used as measurement location for a probe to measure the response of the switching network (404), without the response of the antenna, as explained below.

In some examples, the slot antenna (406), may include a slot (806) having an increased width (812) on both sides of the feed (808) (FIG. 8A). This can be preformed to increase the bandwidth of the slot antenna, since the slot antenna may have limited bandwidth on its own. The end of the feeding strip may also be increased to form a wideband stub (808a), and further increase the bandwidth of the antenna.

The shape of the slot (806) may be varied. For example, it may be a bow-tie slot (FIG. 8E), or the variant shapes shown in FIG. 8F. In general, slot shapes with a smooth increase of the slot width exhibit enhanced response. In at least one example, the hour glass slot shape design (right most design) in FIG. 8F, may exhibit the best performance.

To this end, a slot antenna (406) suitable for use in the present disclosure is preferably designed specifically for the frequency band to be used (e.g., microwave signals), and specifically for use in contact with biological tissues. In at least one example, to feed the slot (806), a complete magnetic coupling is used to minimize the parasitic inductance that a direct connected feed exhibits. By avoiding the parasitic induction, this increases the frequency bandwidth for which the feeding performance is acceptable.

As exemplified in FIG. 8A, the slot antenna (406) may be assembled as a discrete component. This may be preformed to simplify the PCB stack-up. Examples of discrete components are shown in FIG. 8A using PCB technology and 8B representing an example of the bowtie made on a ceramic cap. Such a discrete component can be soldered onto the PCB like any other components as shown in FIG. 8C. FIGS. 8D and 8E exemplify the same slot antenna (406) design, but fully integrated within the PCB and mounting substrate (502).

Referring back to FIGS. 8A-8B , to allow for proper functioning of the antenna array (102), the closely-proximity slot antenna elements (406) require shielding from neighbouring antennas. That is, each slot antenna element (406) operates as much as possible independently from the surrounding antennas (406), and particularly the direct neighbouring antennas.

Accordingly, in some examples, each slot antenna (406) further includes a shielding interface (850) to allow for electromagnetic shielding between adjacent and/or neighbouring slot antennas, and to minimize coupling between antennas. In turn, the antenna array (102) can be designed with a plurality of closely spaced antennas (e.g., densely packed antennas), with each antenna element (406) acting independently rather than interacting. As disclosed, this allows the use of antenna arrays that include many antennas, that can be used for generating many signal paths and providing high-resolution scanning of tissue via sufficient spatial sampling. The shielding can also improve antenna performance on its own and thereby recording cleaner signals.

In particular, the shielding requirement includes two aspects: (i) first, the antenna response of the antenna should not be influenced by its neighbours, and (ii) secondly, the feed network should prevent signals from adjacent antennas from corrupting the signal of interest.

In FIG. 8A (as well as FIGS. 8D and 8E), the shielding interface (850) comprises a dielectric filling material (812) surrounding the feed (808) and feeding via (810), and a ring of grounding vias (814) individually fabricated around, and within, the outer perimeter of the dielectric cavity (812) (e.g., each extending between upper side (802a) and lower side (802b)).

In some examples, each grounding via (814) may be metallised. These grounding vias (814) therefore produce a metallic wall around the antenna and its feed, which reduces interaction between neighbouring antenna elements. To this end, the feeding via (810) may also be formed within the dielectric filling material (812).

In the example of FIG. 8A, a metallic backing (814′) can also be provided, as part of the shielding interface (850), and coupled to the PCB. The metallic backing (814′) can have a small aperture (816), for allowing feeding via (810) to electrically connect to the PCB.

In other examples, the shielding interface (850) can comprise a continuous, or partially continuous, metallic outer cover (e.g., a cylindrical outer cover, in place of grounding vias (814)), surrounding the feed (808) and via (810). In these examples, no filling material (812) is necessary, and feed (808) and feed via (810) can be surrounded by an air medium disposed within the metallic cover.

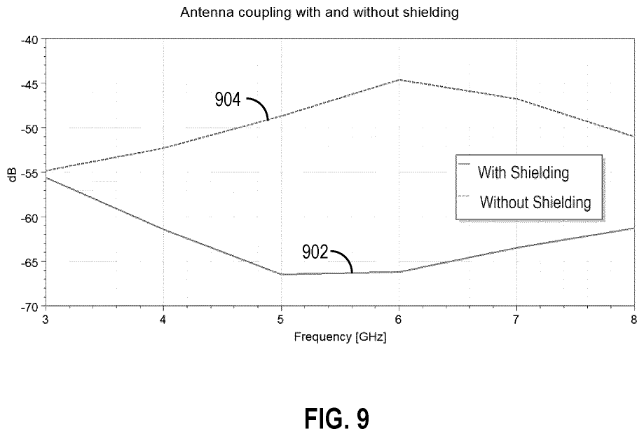

To this end, FIG. 9 shows a plot (900) of the example effect of shielding the slot antenna, within an example antenna array (102), and using shielding interface (850). Plot line (902) provides the antenna coupling without shielding, while plot line (904) provides the antenna coupling with shielding. As shown, with shielding, an improvement of up to 20 dB is provided, to minimize coupling.

Maintaining enough isolation in the feed networks can also be achieved by selecting microwave switches, in switching networks (404), with large isolation between ports and careful PCB routing.

While the slot antenna (406) is exemplified as having a cylindrical shape, in other example, the antenna (406) can have any other shape. For instance, as exemplified in FIG. 8C, the slot antenna (406) can have a hexagonal shape. This can allow for the slot antennas (406) to be positioned side-by-side in antenna array (102) (e.g., a honeycomb pattern), thereby increasing the spatial density in the antenna array (102) and providing for additional signal paths between transmitting and receiving antenna pairs.

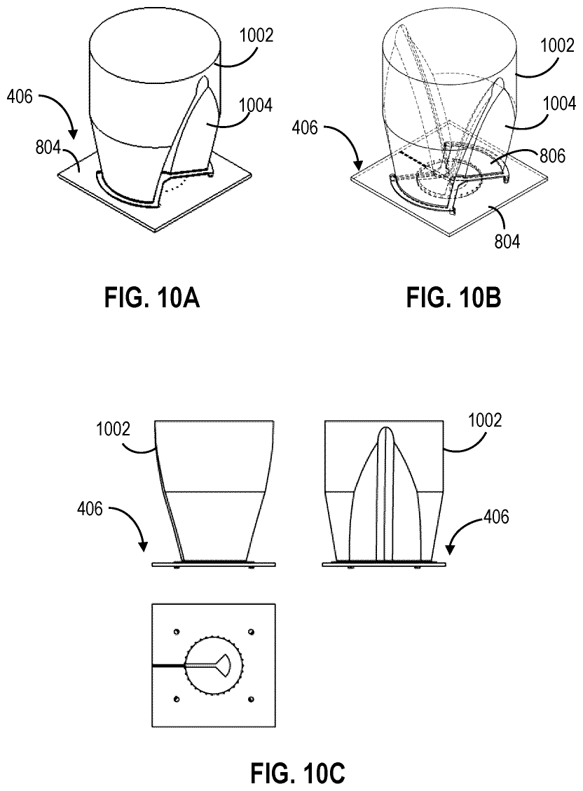

In some examples, as shown in FIGS. 10A-10C, a waveguide structure (1002) may be coupled to the slot antenna (406), e.g., coupled to the upper surface (802a) and on top of substrate (804). The waveguide structure (1002) can include one or more ridges placed on the slot antennas (406), and can overlay over the slot (806) (FIG. 10B). The waveguide (1002) can function to increase directivity and gain of the antenna.

The section of the ridged waveguide (1002) may be of any geometric shape. For instance, as shown, the waveguide (1002) may have a conical structure.

In at least one example, the waveguide structure (1002) has, at the very least, a conductive material on its circumference or outer surface. The ridges (1004) may be oriented into the waveguide space and opposite to each other, and complementing the slot (806) design shape. The ridges may be in close proximity to each other at the slot location and flare externally as it extends in the ridged waveguide. In at least one example, the ridges (1004) include a conductive coating, and may improve the transition from the slot (806) to the waveguide (1002).

The ridged waveguide may be made of a material with higher permittivity value than standard or conventional waveguide material, such as an engineered plastic or ceramic.

### (ii.) Dielectric Material Covering.

While contacting the antenna arrays (102a), (102b) directly with the biological tissues (412) has a positive effect (e.g., signals are coupled into the tissues rather than experiencing reflections), direct contact between the antenna metallization and the tissues may negatively impact the efficiency of the antenna. Accordingly, in some examples, a non-electrically conductive (dielectric material) cover is placed between the antenna arrays (102) and the biological tissue, to minimise the reduction of efficiency.

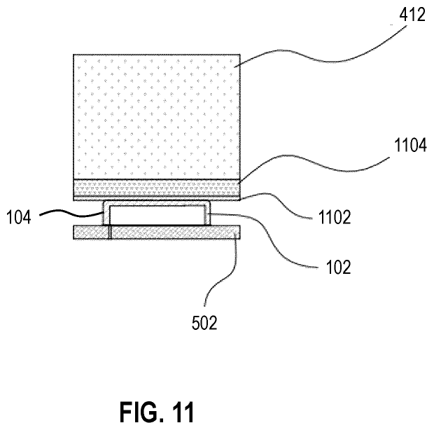

For instance, as exemplified in FIG. 11, each antenna array housings (104) may be covered by a thin dielectric (non-electrically conductive) material (1102) (see also FIGS. 4A and 4B). The dielectric material (1102) is coupled to, and overlaid over (e.g., mounted to), the antenna array (102), and directed toward the scanning region (158).

When biological tissue (412) is inserted into the scanning region (158), the dielectric material (1102) is disposed between a respective antenna array (102) and the biological tissue (412) (FIGS. 4A and 4B). For example, the dielectric material (1102) contacts and engages the skin (1104) around the scanned biological tissue. The dielectric cover material (1102) may comprise any non-electrically conductive material, such as any suitable polymer or ceramic.

More generally, the dielectric cover material (1102) is formed of low permittivity material and is sufficiently thin to promote coupling, and avoid signals leaking around the biological tissues (412).

In at least one example, the dielectric cover material (1102) is formed of material having a relative permittivity below 5, and a maximum thickness of 2 mm, and in some examples, a relative permittivity around 2.5 and thickness below 0.5 mm. As used herein, the thickness of the material is defined along the axis (150a).

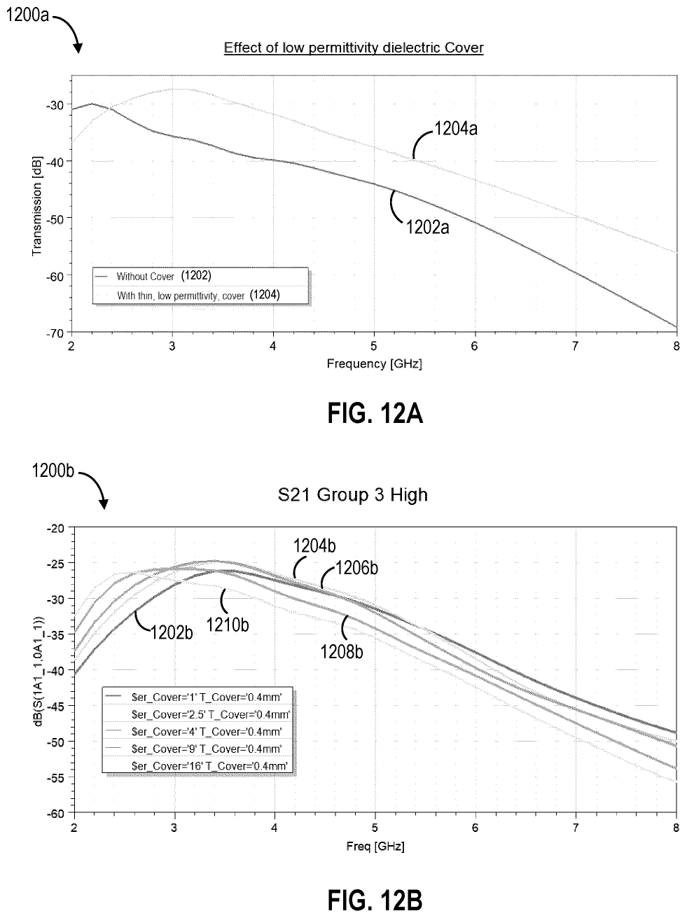

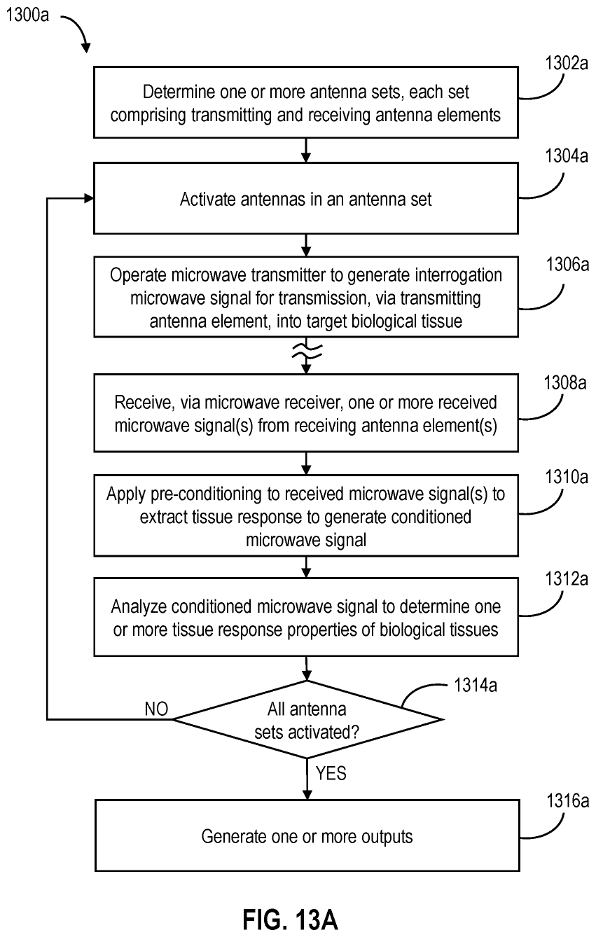

FIG. 12A shows an example plot (1200a) illustrating the effect of a cover (1102) made of material with relative permittivity of 2.5 and a thickness of 0.4 mm. A comparison is provided for antenna arrays (102a), (102b) without a cover (plot line (1202a)), versus antenna arrays (102a), (102b) with a dielectric cover (plot line (1204a)).

As shown in plot (1200a), as compared to antenna arrays (102) without a dielectric cover (1102), an increase of between 6 and 12 dB in transmission coefficient is observed for the antenna arrays (102a), (102b) with the dielectric cover (1204a). An additional 10 dB loss in the arrays without a cover potentially reduces the signal strength to a point where the signal is unrecoverable, e.g., from the thermal noise. For reference, compensating for 10 dB attenuation of a signal requires the measurement time to be increased by a factor 10 to lower the noise floor. This is not practical for biomedical imaging, as the scan time may increase from less than 30 seconds to 5 minutes.

FIG. 12B shows plot (1200b) that compares antenna arrays (102a), (102b) with dielectric covers (1102) having 0.4 mm thickness, and different values of permittivity. These include a permittivity of 1 (1202b), 2.5 (1204b), 4 (1206b), 9 (1208b) and 15 (1210b). It is observed that a dielectric cover (1102) with higher permittivity leads to a weaker transmission at higher frequencies. In particular, signals propagating through tissues experience greater attenuation at higher frequencies. Greater transmission at higher frequencies is critical for enabling high sensitivity.

### V. Example Method(s)

The following description relates to various example methods for imaging biological tissue, in accordance with disclosed embodiments.

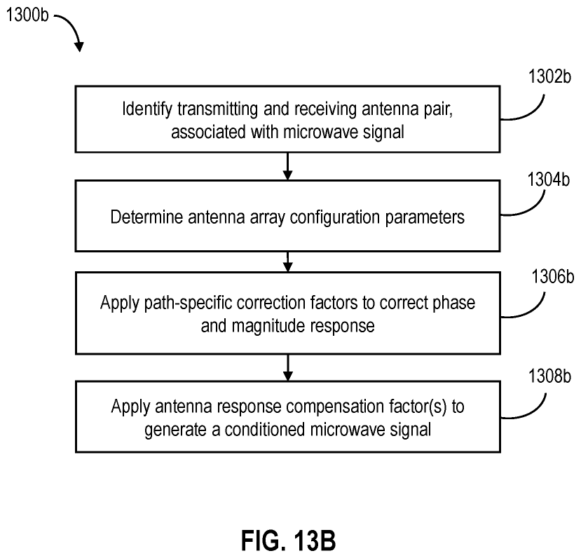

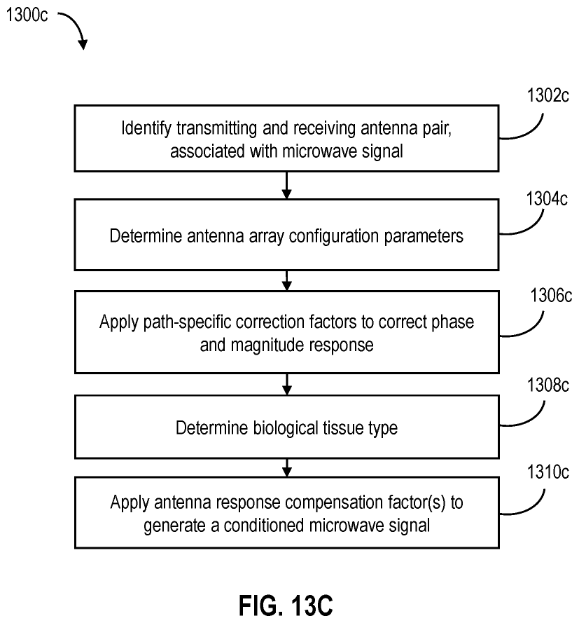

Each of the described methods (1300a)-(1300c) (FIGS. 13A-13C) can be executed by controller (418). In other examples, the methods may be executed by controller (418) alone, or in combination (e.g., conjunction) with one or more external computing devices (e.g., servers).

### (i.) Overall Method.

FIG. 13A shows a process flow for an example method (1300a) for imaging biological tissue (412), e.g., inserted in the scanning region (158) between two antenna assemblies (402a), (402b) (FIG. 4A or FIG. 4B).

At (1302a), one or more antenna sets are selected for imaging the target biological tissue.

For example, as shown in FIG. 5A, each antenna set (512) includes one transmitting antenna element (406a), and one or more corresponding receiving antenna elements (406b) associated (e.g., assigned to) the transmitting antenna element (406a). Accordingly, each antenna set (512) include a plurality of antenna pairs, each antenna pair comprising the transmitting antenna (406a) and one of the receiving antennas (406b).

In some examples, the transmitting antenna (406a) is selected within a first antenna array (102a), while the one or more receiving elements (406b) are selected within a second antenna array (102b). It is also possible that the antennas are selected within the same array.

More generally, as shown in FIG. 5A, each antenna set (512) can include one or more receiving antennas (406b) (and in some examples, two or more receiving antennas (406b)), collectively forming a receiving antenna group (502). Accordingly, within each antenna set (512), each transmitting antenna (406a) is therefore associated with a group (502) of receiving antenna elements (406b). The group of receiving antennas (502) are the antennas positionally located to receive the most relevant transmitted microwave signal, both in term of signal strength and geometrical path.

As provided herein, the purpose of defining the receiving antenna group (502) is to simplify and expediate signal processing and analysis. In particular, to simplify complexity of signal analysis, and therefore increase system computational speed, the system can disregard signals received by other antennas elements (406b) in the receiving antenna array (102b), located outside the receiving group (502).

In some examples, the group of receiving antennas (502) is determined by initially identifying an opposing receiving antenna elements (406b) that is directly opposite the transmitting antenna element (406a) (e.g., along axis (150a) (FIG. 4A or FIG. 4B)). Further, a pre-defined number of receiving antennas (406b), including and neighbouring that opposing receiving antenna (406b), are selected for that receiving antenna group (502). In some examples, antenna elements (406), located within 60 mm radius of the opposing receiving antenna (406), are included within the group (502).

In other examples, each antenna set (512) simply includes a single transmitting and receiving antenna (406). Accordingly, the antenna group (502), in this example, includes only a single receiving antenna (406).

In some examples, at (1302a), a plurality of antenna sets are determined for different transmitting antennas (406a), in the transmitting antenna array (102a). For each transmitting antenna (406a), a receiving antenna group (502) is associated with that transmitting antenna (406a) to define a respective antenna set (512) for that transmitting antenna (406a). As between different antenna sets (512), the receiving antennas (406b) included in each receiving group (502) may overlap.

In at least one example, at (1302a), an antenna set (512) is selected for each transmitting antenna (406a), in the transmitting antenna array (102b).

To this end, the determination at (1302a) can be performed in various manners. In some examples, the selection of antenna elements in each antenna set (512) is determined manually, in advance (e.g., by a system operator).

At (1304a), antenna elements (406), in one of the antenna sets (512), are activated.

For example, as shown in FIG. 4A, this involves operating the switching network (404a)—associated with the transmitting antenna array (102a)—to couple the transmitting antenna element (406a), in the selected antenna set (512), to the microwave transmitter (416a).

Further, switching network (404b)—associated with the receiving antenna array (102b)—is operated to couple each of the receiving antenna elements (406), in the associated group of receiving antennas (502), to the microwave receiver (416b).

In some examples, the switching network (404b) is operated to couple one receiving antenna (406b) at a time, in group (502), to the microwave receiver (416b). In these cases, acts (1306a)-(1312a) are iterated, whereby—in each iteration—a different receiving antenna (406b) is coupled to the microwave receiver (416b). In other examples, all of the receiving antennas in group (502), are concurrently (e.g., simultaneously) coupled to the microwave receivers (416b).

Otherwise, in the example of FIG. 4B—if switching networks (404) are not provided, then activating the antenna elements, in an antenna set (512), at (1304a), involves operating the microwave transmitters (416a) and microwave receivers (416b), that are associated with (e.g., coupled to), the corresponding antenna elements that require activation. Again, with respect to the receiving antennas (406b), this may involve operating one microwave receiver (416b) at a time, or operating multiple receivers concurrently.

At (1306a), the microwave transmitter (416a)—e.g., alone, or within microwave transceiver (416)—is operated to generate an interrogation microwave signal. The interrogation microwave signal is routed to the respective activated transmitting antenna element (406a). The activated transmitting antenna (406a) accordingly transmits the interrogation microwave signal (414), such as to allow the microwave signal to pass through the biological tissue (412), disposed within the scanning region (158) (FIGS. 4A and 4B).

At (1308a), the microwave signal (414) passes through the biological tissue (412), and the group of activated receiving antennas (502) each receive a corresponding received microwave signal. As noted above, the receiving antennas (502) may receive the microwave signals concurrently, if they are activated concurrently. In other examples, if the microwave receivers (406b) are activated one at a time, then each microwave receiver (406b) receives the microwave signal from the transmitting antenna, upon its activation.

To this end, as shown in FIG. 5A, each of the activated receiving antennas (406b)—within the group (502)—receives a corresponding received microwave signal. Each received microwave signal corresponds to the interrogation microwave signal, at a given activated receiving antenna (406b), and after passing through the biological tissue.

In this manner, a plurality of microwave signal paths (508) are defined between each pair of the activated transmitting antenna (406a) and one of the activated receiving antenna (406b).

Each received microwave signal is then routed, from the receiving antenna (406b), to the microwave receiver (416b), for recording and further processing. In FIG. 4A, the microwave signals are routed to the microwave receiver (416b), via the switching network (404b). In FIG. 4B, the microwave signal is directly routed to the microwave receiver (416b), associated with the receiving antenna (406b).

At (1310a), each received microwave signal—from each activated receiving antenna (406b)—is pre-conditioned to generate a corresponding conditioned microwave signal.

As provided in FIGS. 13B and 13C, the signal pre-conditioning is used to correct for various error-inducing factors, including phase and magnitude response correction as well as correcting for antenna radiation characteristics.

More generally, the signal pre-conditioning is applied to each received microwave signal, from each activated receiving antenna (406b). As explained in FIGS. 13B and 13C, the pre-conditioning applied to a received microwave signal is based on the properties of the signal path (508) associated with that received microwave signal.

As explained previously, an appreciated technical advantage of defining each antenna set (512), to include only a group (502) or receiving antenna elements (406b), is to provide for computationally efficiencies at act (1310a). That is, the signal pre-conditioning needs only be applied to microwave signals received by a select group of activated receiving antennas (406b), positioned to receive the microwave signal most strongly (i.e., rather than every signal received by every antenna in the receiving antenna array).

At (1312a), each of the conditioned microwave signals is analyzed to determine one or more tissue response properties (e.g., tissue electrical response properties), of the scanned biological tissue (412). In some examples, one or more tissue features are determined from the tissue electrical response properties (e.g., water content (and/or state of the water such as bound water or free water), density of the biological tissues).

At (1314a), it is determined whether all antenna sets (512) have been activated. If not, the method returns to act (1304a) to activate the next antenna set. That is, activating the next transmitting antenna (406a), and associated group (502) of receiving antennas (406b).

As used herein, in each iteration of method (1300a), using a different activated antenna set (512) corresponds to a single “scan” of the biological tissue. Accordingly, the method is iterated until all antenna sets have been activated, and tissue response properties are determined at (1312a) based on each scan.

As explained previously, the ability of the system to conduct multiple scans using different combinations of antenna sets (512), allows generating different signal paths (508) throughout different areas of large volume biological tissue (412) (FIG. 5B), and quickly acquiring corresponding tissue response data. This enhances the data resolution and accuracy of the system. The ability to interrogate and scan the biological tissue with a large number of signal paths is facilitated by using the slot antenna design, which allows for densely packed antennas (FIG. 7). In particular, the use of the slot antenna design allows each receiving antenna group (502) to include a large number of receiving antennas (406b), that are positioned to receive the microwave signals—along different signal paths—strongly, and therefore providing useful data.

As noted above, in some examples, the antenna arrays can be moved laterally and angularly re-positioned (FIG. 3B), to capture data from different areas of the biological tissue. However, this may not be necessary, as the multitude of signal paths-generated in different locations, and travelling along different angles-allows for scanning different regions of large biological tissue, without needing to move the antenna arrays around the tissue.

At (1316a), based on the signal and analysis processing, one or more outputs are generated. The disclosure herein is not limited to the type or form of output. For instance, in at least one example, the tissue response properties are visually represented in one or more output 2D or 3D images (FIGS. 2A and 2B). These images can be displayed, for example, on a display screen (114) (FIG. 1). Display screen (114) can be associated, or otherwise coupled to, the controller (418).

### (ii.) Method for Signal Preconditioning.

FIG. 13B shows a process flow for an example method (1300b) for signal preconditioning to generate a conditioned microwave signal. The conditioned signal includes the unique tissue response, subject to the microwave signal, and otherwise removes extraneous responses. In some examples, method (1300b) is executed during act (1310a), in FIG. 13A.

More generally, method (1300b) is applied for each received microwave signal at (1308a) (FIG. 13A). That is, within a given activated antenna set (1304a), method (1300b) is applied for each microwave signal transmitted by an activated transmitting antenna (406a) and received at a given activated receiving antenna element (406b).

Accordingly, in a single iteration of method (1300a) (FIG. 13A), the method of FIG. 13B is applied independently for each received microwave signal, at each activated receiving antenna element (406b) within the activated antenna set (e.g., in FIG. 5A, each of the signals for each signal path (508)).

At a general level, at (1302b), the system initially identifies the transmitting and receiving antenna pair, associated with the received microwave signal. This allows the system to determine the exact signal path (508) followed by the microwave signal. It also allows the system to determine the types of correction and compensation factors to apply at acts (1306b) and (1308b).

In some examples, the determination at (1302b) is based on the known configuration of: (i) the transmitting switching network (404a), which indicates which transmitting antenna (406a) is associated with the microwave signal; and (ii) the receiving switching network (404b), which indicates which receiving antenna (406b) received the microwave signal. Otherwise, in FIG. 4B, this information is known based on which microwave transmitters/receivers were being operated at a given time instance.

At (1304c), based on the determination at (1302b), the system determines one or more antenna array configuration parameters. As detailed herein, the configuration parameters can be used for performing signal preconditioning at (1306b) and (1308b). In at least one example, the antenna array configuration parameters include the following:
(i) positional configuration of antenna elements on each array—in some examples, this data is stored on the controller's memory (2004) (FIG. 20). Positional configuration information can include the relative offsets (e.g., x, y) of each antenna element on the array, and can also include a vertical (z) offset, such as in the case of a curved antenna array. The positional configuration is not necessarily expressed in cartesian coordinates, e.g., it can also be expressed in polar coordinates. Positional configuration information can also include the orientation of each antenna element on the array using for example an Euler angle representation (e.g., ψ, Θ, φ);
(ii) the separation distance (152) between the antenna arrays (102a), (102b)—in some examples, this predetermined or manually input into the system. In other examples, as shown in FIGS. 4A and 4B, the controller (418) is coupled to one or more distance sensors (422a), which monitor the separation distance (152). For example, the distance sensors may be integrated within the mechanical system (108), and can include magnetic reader measuring location on an encoded magnetic strip to measure the separation distance (152). Other distance sensors known in the art can be used, such as optical line of sight sensors mounted on the housings (104); and
(iii) rotational position of the antenna arrays (102a), (102b) (e.g., along an axis parallel to rotation axis (150b) in FIG. 3B)—a gain, in some examples, this is pre-determined or manually input into the system. In other examples, as shown in FIGS. 4A and 4B, the controller (418) is coupled to one or more rotational sensors (422b), which monitor the rotation of each antenna array (e.g., rotary position sensors, as known in the art). The rotation sensors may be coupled antenna housings (104), for example.

More generally, the antenna array configuration parameters can also be used to determine the signal path properties, associated with the received microwave signal.

To this end, as shown in FIG. 5A, each signal path (508)—defined between the activated transmitting antenna (406a), and each activated receiving antenna (406b)—may have different signal path properties.

Signal path properties include, for example, the signal path length (e.g., between the associated transmitting and receiving antennas), incident azimuth on the receiving antenna, and path offset relative to other antennas in the receiving antenna array.

Here, it will be understood that signal path properties are affected by the positional configuration of antenna elements. That is, different arrays may be defined by different spacing offsets between the antenna elements. That is, the antenna elements (406) on different arrays (102), may be arranged differently around the array. The positional arrangement, of receiving antenna elements on the array, may effect the signal path properties for each receiving antenna element.

Additionally, the signal path properties are affected by the separation distance (152) (FIG. 5A) between the antenna arrays, which vary with at least the signal path length and azimuth. Further, the rotation of the antenna arrays (FIG. 3B) can also vary the signal path length and azimuth.

Accordingly, in at least one example, determining the signal path properties at (1304b) involves initially determining the separation distance (152) and/or rotational positional of antenna arrays (102a), (102b).

As explained herein, as the system is able to accommodate for different separation distances and rotational positions of antenna arrays—the system is, in turn, able to accommodate for different biological tissues, and generate appropriate conditioned microwave signals. For example, despite the fact that two individuals may have two different sizes of breasts—and therefore, the separation and rotational configuration of the antenna arrays require adjustment—the system accommodates for these patient-specific adjustments, and their factors that impact on the signal path properties.

Based on the determinations at (1302b) and (1304b), at (1306b), one or more path-specific corrections are applied to the microwave signal to correct for the phase and magnitude response and generate a corrected microwave signal. As explained herein, the phase and magnitude corrections are applied to correct for the effects of the measurement hardware, and is applied for both the transmitting and receiving antenna pathways.

Subsequently, at (1308b), a path-specific antenna response compensation factor is applied to the corrected microwave signal, to generate the corresponding conditioned microwave signal. The path-specific compensation factor removes the antenna response from the corrected microwave signal.

Accordingly, the conditioned microwave signal is representative of the biological tissue response, and removes the effects of the measurement in the hardware. This tissue response can then be used to determine various tissue properties with more accuracy, as explained herein.

As explained previously, acts (1306b) and (1308b) may be performed via digital signal processing, using the processor of controller (418) (or any other external computing systems).

To this end, as explained herein, a unique aspect of the disclosed signal preconditioning method, is that it relies on applying pre-determined reference datasets pertaining to correction and compensation factors. The use of reference datasets greatly simplifies the signal preconditioning process, and allows for higher computational and processing speeds when filtering the signals digitally. In turn, this reduces the amount of time required to generated desired outputs.

Acts (1306b) and (1308b) are now explained herein, in greater detail.

### (ii.a) Phase and Magnitude Correction.

At (1306b), phase and magnitude response correction is applied to a received microwave signal.

More generally, the purpose of act (1306b) is to isolate and remove, from the received microwave signal, the responses generated along: (i) a transmission pathway extending between microwave transmitter (416a) and the specific activated transmitting antenna (406a) associated with the microwave signal, and, (ii) a receiving pathway extending between the activated receiving antenna (406b), associated with the received microwave signal, and the microwave receiver (416b). In this manner, the microwave signal does not include extraneous responses generated by the hardware.

In some examples, one or more path-specific correction factors are applied to the microwave signal, including: (i) a first phase correction factor for the transmission pathway, (ii) a first magnitude correction factor for the transmission pathway; (iii) a second phase correction factor for the receiving pathway; and (iv) a second magnitude correction factor for the receiving pathways. It is understood that the correction factors are referenced as “path-specific”, as the correction factors vary for different transmitting/receiving antennas.

To this end, the correction, at act (1306b), can be applied using a pre-defined set of path-specific correction factors.

For example, controller memory (2004) (FIG. 20) can store a reference dataset of correction factors (e.g., a reference look-up table) comprised of known magnitude and phase correction factors for each transmission and receiving pathway in the system (400).

For instance, the reference dataset can store different phase and magnitude corrections factors, for each transmission pathway defined between, (a) the microwave transmitter (416a), and (b) each transmitting antenna element (406a) in the transmitting antenna array (102a). Further, the dataset can also store phase and magnitude correction factors for each receiving pathway defined between: (a) each receiving antenna element (406b), in the receiving antenna array (102b), and (b) the microwave receiver (416b). In at least one example, the reference dataset can also store different phase and magnitude correction factors, for different frequencies of microwave signals, as these factors can be frequency dependent.

Accordingly, while executing (1306b), the system can identify—based on the transmitting and receiving antennas associated with the microwave signal (1302b)—the specific transmission and receiving pathways associated with these antennas. The pathways are then used to reference the correction factor dataset, and in turn, identify the “path-specific” correction factors, associated with each those pathways, and for both the magnitude and phase correction. If multiple frequencies of microwave signals are used, then the system can also identify the known microwave frequency being transmitted, and identify the correction factors associated with that frequency.

In at least one example, the reference correction factor dataset is generated in advance by testing the system (400) using well-known vector network analyzer techniques.

For example, at a time prior to scanning the biological tissue, the path response between the microwave transceiver (416) and each antenna element (406) is measured, e.g., using a 2-port vector network analyzer. For instance, the network analyzer is connected, and re-connected, between the main port of the antenna array and the feed location of each antenna element (406). The measured path responses (e.g., magnitude and phase) between the microwave transmitter and/or receiver, and each antenna element (406), are then recorded as path-specific correction factors associated with each antenna element in the fabricated array (102) (e.g., as transfer functions, as known in the art). In some examples, the dataset includes antenna element identifiers, and associated transmission and/or receiving pathway correction factors. This can also be repeated for different signal frequencies, to generate frequency-dependent correction factors.

To this end, if a 2-port vector network analyzer is used to determine path responses, various techniques can be employed to accurately measure the path response, as known in the art. For example, the correction factors may be determined as a simple response calibration between the transmit and receive ports, or otherwise determined using open-short-load calibration, transmission-reflect-through calibration, or any other calibration techniques as known in the field.

In some examples, a specialized insertable probe is used to connect the network analyzer to the antenna feed location. Alternatively, a non-insertable probe (e.g., another antenna), may be used to externally couple with the antenna element (406), in place of an insertable probe.

In other examples, the path responses may be measured via one or more measurements of one or more reference materials or objects. Knowing the response of the reference material, the path response for each antenna pair is then de-embedded from the measured response using scattering matrix manipulation.

Accordingly, once act (1306b) is performed, the received microwave signal is corrected for magnitude and phase distortions. In turn, a corrected microwave signal is generated, with corrected phase and magnitude properties.

### (ii.b) Antenna Response Correction.

At (1308b), the antenna response is removed from the received microwave signal. The antenna response includes antenna radiation characteristics, including: (i) the antenna gain, and (ii) antenna phase center.

### Antenna Gain.

As known in the art, antenna gain defines the ability of a transmitting antenna element (406a) to convert input power into a radiated microwave signal, or otherwise, the ability of a receiving antenna element (406b) to convert a received microwave signal into output electrical power.

In an ideal case, a transmitting antenna (406a) converts one hundred percent of the supplied power into a transmitted microwave signal. Further, a receiving antenna (406b) converts one hundred percent of the received microwave signal into output electrical power, and irrespective of the direction of incidence of the received microwave signal onto the received antenna.

In operation, however, the transmitting and receiving antenna elements do not exhibit perfect antenna gain. For example, the transmitting antenna element (406a) may not convert all received input electrical power (e.g., from microwave transmitter (416a)), into the transmitted microwave signal. Likewise, the receiving antenna element (406b) may not convert the received microwave signal, into output electrical power. Each antenna element will exhibit some degree of loss.

Importantly, with respect to the receiving antenna element (406b), the antenna gain is affected by a non-isotropic radiation pattern. More particularly, the radiation pattern from the transmitting antenna (406a)—in the activated antenna set (512) (FIG. 5A)—is often non-isotropic, such that microwave signals of the same magnitude and incident on receiving antennas from different directions, may result in different received powers at different receiving antennas. Therefore, in order to properly extract the tissue response, different “path-specific” (508) antenna gain compensations are applied to different microwave signals, based on their unique signal path properties.

### Phase Center.

In addition, or in alternative to antenna gain compensation—the antenna response compensation at (1308b) can also include phase center compensation.

The phase center of a transmitting antenna (406a), is a point in space where the radiated signal appears to originate. This point is not necessarily at the physical center or edge of the antenna, or even on the radiating element itself. Hence, the removal of the antenna response also includes the compensation for the distorted (e.g., non-ideal) location of the phase center of the transmitting antenna (406a). The phase center compensation is compensated for in the same way as the gain, as it also path (508) specific.

### Antenna Response Compensation Factors.

In view of the foregoing, at (1308b), for a given corrected microwave signal, generated at (1306b)—antenna response compensation factors are applied to that signal, to generate the conditioned microwave signal.

In at least one example, applying the antenna response compensation factors include applying both: (i) the antenna radiation gain compensation factor; and (ii) phase center location compensation factor, which are function of frequency, antenna structure and the signal path (508) properties (e.g., incident azimuth and elevation).

In at least one example, the disclosed system can include a reference compensation factor dataset (e.g., a reference database)—e.g., stored on controller memory (2004)—which includes pre-determined gain and phase center compensation for each antenna structure, in system (400). In some examples, gain and phase center compensations are associated with different azimuth and offset properties, of receiving antennas relative to a transmitting antenna. In this manner, at (1308b), the correct compensation factors are selected in the reference dataset, based on the specific signal path properties identified at (1304b).

In some examples, because the compensation factors also vary based on signal path properties—the reference compensation factor dataset can also include different compensation factors (e.g., gain and phase center) for different combinations of separation distances (152) (e.g., between antenna arrays) and/or rotational orientations of antenna arrays (e.g., as determined based on the antenna array configuration parameters, at (1304b)) Accordingly, at (1308b), the correct compensation factors are selected in the reference dataset, based on the spacing distance and path orientation, determined at (1304b).

In other examples, the reference dataset can store the compensation factors in association with different signal path lengths and incident azimuths, as well as signal frequencies.

It has also been appreciated that antenna response compensation also changes significantly when the antenna arrays (102a), (102b) are in contact with different biological tissues. This includes varying responses between different types of tissue (e.g., breast versus other tissue), as well as for the same tissue between different individuals.

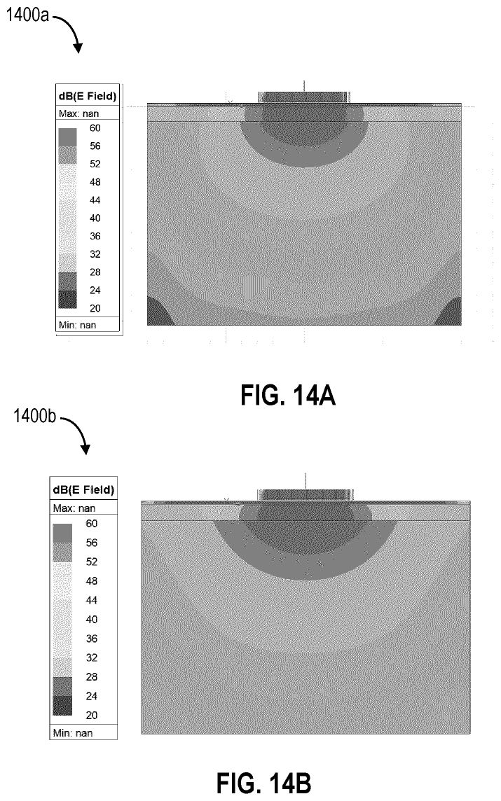

FIGS. 14A and 14B illustrate, with respect to the antenna gain characteristics, the effect of different biological tissue. FIG. 14A is a nearfield radiation intensity pattern (1400a) in tissues that are predominantly fatty, while FIG. 14B is a nearfield radiation intensity pattern (1400b) in tissues with greater glandular content. A change in the field intensity, of the antenna gain, is clearly observed between the two radiation patterns.

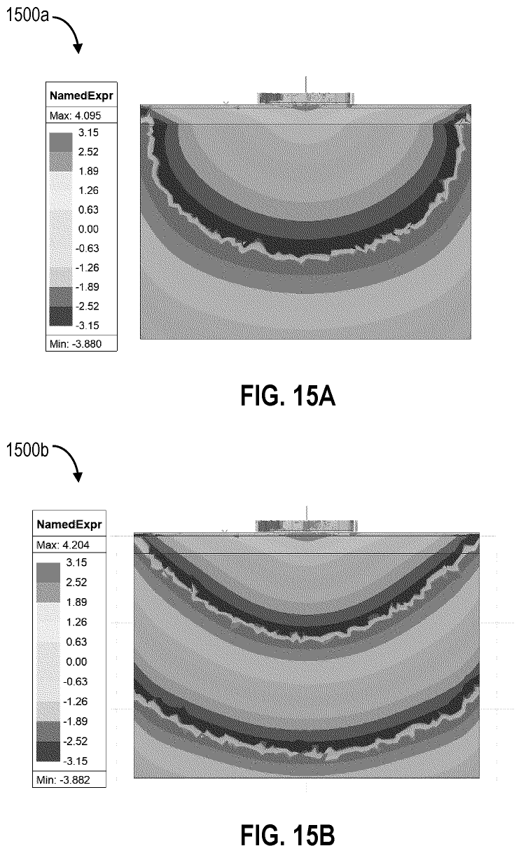

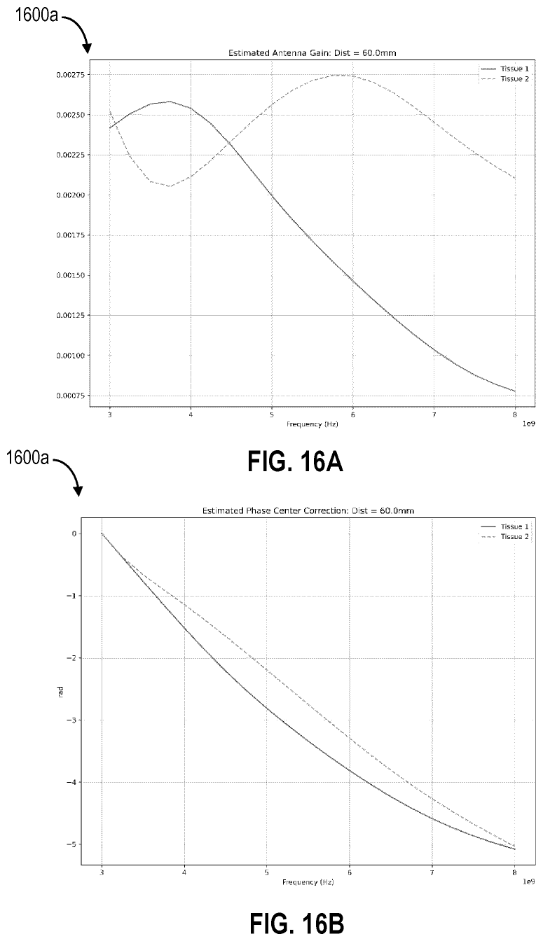

FIGS. 15A and 15B illustrate, with respect to the phase center characteristics, the effect of different biological tissue. In particular, the phase is also plotted in the vicinity of the antenna for different tissue properties. FIG. 15A is the nearfield phase pattern (1500a) in tissues that are predominantly fatty, while FIG. 15B is the nearfield phase pattern (1500b) in tissues with greater glandular content. Again, clearly observed is the change in shape of the phase which translates into a modification of the phase center depending on the observation location and/or orientation with respect to the antenna.

In view of the foregoing, a technical challenge with using the disclosed antenna array configuration (e.g., FIGS. 4A and 4B), is applying antenna response compensation having regard to different biological tissue being scanned.

To accommodate for this, the compensation factor reference dataset (e.g., reference look-up) can also include different, pre-determined compensation factors for different biological tissue types.

In some examples, the reference dataset can include multiple sub-datasets (e.g., sub-reference lookup) for each biological tissue type. Each sub-dataset, associated with each biological tissue type, can then include reference data regarding tissue-specific antenna response factors, including: (i) tissue-specific compensation factors determined for each antenna element; and (ii) tissue-specific compensation factors determined for different antenna array configuration parameters (e.g., spatial distances between the antenna arrays and/or rotational orientations of the antenna arrays).

Accordingly, at (1308c), in some examples, the system can initially determine the type of biological tissue being scanned. Based on this, the system can access the reference sub-dataset associated with that biological tissue type, and determine the correct tissue-specific antenna response compensation factors, as noted above.

In at least one example, the antenna response compensation factors comprise transfer functions.

In at least one example, the reference dataset of compensation factors is generated, in advance, through simulation of the antenna design using industry standard simulation tools, and with different biological tissues. Example simulation criteria are explained below.

Specifically, simulations of transmission coefficient between antennas in contact with tissue models are performed for a range of separation distances between the arrays and/or rotational orientation of arrays. Here, the transmission coefficient encompasses the effect of the antenna transfer function for both the emitting and receiving antenna. The tissue models used may be represented, in the simulation tools, as a set of Debye models describing a range of tissue to be expected. For each tissue model and each signal path (508) orientation, the transmission coefficient is simulated for multiple separation distances (152) and/or rotational antenna orientations. This allows a model (polynomial that is a function of separation distance and/or rotational antenna orientation) to be developed that describes required correction for a particular tissue arrangement of antennas (path) and a specific material (tissue model).

Alternatively, instead of simulating the transmission coefficient between two antenna pairs, these detailed characterizations can be performed through simulation of an antenna transfer function from the antenna feed to a range of observation points in space. The locations of the observation points in space need to match the trajectory of the path for which the compensation is applied. The actual antenna response compensation is then calculated by combining the antenna transfer function for the emitting and receiving antenna. Doing so allows for the use of different tissue specific correction factors for each antenna element. Other example methods can also be employed.

In addition, or in the alternative, reference compensation factors may be measured, in advance, by measuring transmission coefficients between two antennas over a range of path directions, and different biological tissues.

For example, it is possible to conduct measurements of a bladder filled with liquids representing a range of biological tissues and at different separation distances (152) to provide compensation factors, instead of simulation. The compensation factors for the antenna radiation characteristics are then selected based on tissue properties estimated for each individual scan.

To this end, FIG. 16A shows a plot (1600a) of antenna gain compensation for two different tissue types, and for a signal path having the same signal path properties. FIG. 16B shows a plot (1600b) for phase center compensation for two different tissue types against frequency, and for a signal path having the same signal path properties.

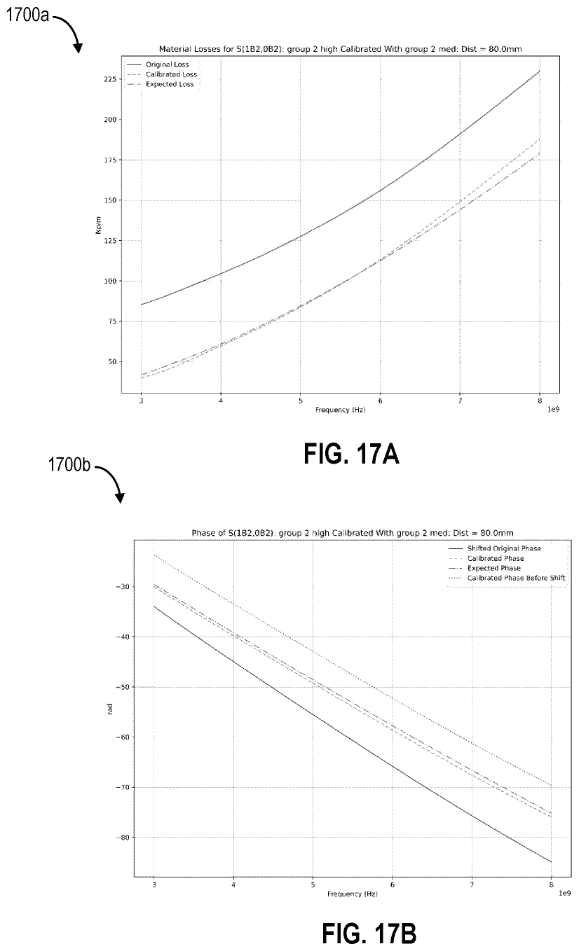

FIGS. 17A and 17B show plots (1700a), (1700b) illustrating the effect of correcting the original signal attenuation and phase shift using the compensation for both the gain and phase center. In particular, these plots show the resulting correction using the gain and phase centre compensation.

In at least one example, as the number of possible signal paths may be much larger than available compensation factor data—if a compensation factor is not pre-determined for a given signal path (for a given biological tissue), the compensation factors may be interpolated and extrapolated using techniques such as fitting a polynomial to available data. The resulting model allows for obtaining correction factors for a wide range of signal paths, with different signal path properties.

In some embodiments, the correction of the antenna radiation characteristics is performed equally for all frequency points, individually for each frequency point, or performed equally for some frequency points and individually for other frequency points.

Accordingly, the key to compensating for gain and phase is detailed characterization of the antenna response in a range of materials with properties corresponding to expected properties of tissues. By removing the antenna characteristics from the measurement, the response of the tissues remains, which can be used to determine tissue response properties.

### (ii.c) Determining Prediction for Biological Tissue Type.

FIG. 13C shows another process flow for an example method (1300c) for signal preconditioning, to generate a conditioned microwave signal. Method (1300c) is executed during act (1310a), in FIG. 13A.

In method (1300c), acts (1302c), (1304c), (1306c) and (1310c) are analogous to acts (1302b), (1304b), (1306b) and (1308b), respectively, of method (1300b). However, method (1300c) also includes act (1308c), which involves determining the type of biological tissue being scanned, prior to applying the tissue-specific antenna response correction.

More particularly, in various cases, the exact biological tissue (412) (FIGS. 4A and 4B), inserted within scanning region (158) may not be known in advance. Accordingly, at (1308c), the system can predict in real-time, or near real-time, the biological tissue type. In turn, the system can apply the correct antenna response compensation factors, associated with that biological tissue type.

In at least one example, to determine the biological tissue type at (1308c), an initial determination of tissue response properties is made. This initial determination of tissue response properties is performed without the benefit of using a conditioned microwave signal that includes the antenna response compensation, and is only meant to provide a prediction of the biological tissue type.

In some examples, a measured tissue response property can correspond to time delay properties of microwave signals, travelling through the biological tissue. An example method for determining various tissue response properties, including time delay, is discussed in the next section.

To this end, a reference tissue properties dataset (e.g., stored on controller memory (2004) (FIG. 20)) can store different reference tissues response properties, for different types of biological tissues. Accordingly, the system maps the determined tissue response properties to the reference tissue properties, to determine the biological tissue with the closest properties.

In at least one example, the reference tissue response properties are generated via computer simulations. For example, simulations are generated with transmit and receive antennas in contact with different biological tissues to predict their tissue response properties. The database may then store a reference lookup of different tissue types, and their predicted tissue response properties. Example simulation criteria is explained further below.

### (iii.) Determining Tissue Response Properties.

The following is a discussion of example tissue response properties of the target biological tissue, that can be determined at act (1312a) (FIG. 13A), based on analyzing the conditioned microwave signal.

In some examples, the tissue response properties include various electrical response properties of the scanned biological tissue and include, as non-limiting examples, permittivity, conductivity, attenuation, phase constant, and dielectric relaxation time properties of the tissues.

For example, a method for determining permeability from time delay properties is described in J. Bourqui and E. Fear, “System for Bulk Dielectric Permittivity Estimation of Breast Tissues at Microwave Frequencies”, IEEE Transactions on Microwave Theory and Techniques, Vol. 64, No. 9, September 2016, which is incorporated herein by reference. Based on the time delay properties, permittivity and conductive properties can be determined, as described in this reference.

In at least one example, the determination of tissue response properties incorporates the separation distance (152) between the activated transmitting and receiving antennas.

In some examples, the tissue response properties are determined in the frequency domain, and in turn, provide frequency dependent electrical properties of the tissue. The frequency dependent electrical properties may include, as non-limiting examples, permittivity, conductivity, attenuation constant, phase constant, or parameters of a dispersive property model. The dispersive property model can include the Debye model that includes, static permittivity, infinite permittivity, static conductivity and dielectric relaxation time. Frequency domain calculations of tissue properties may be based on the phase and magnitude response of the frequency domain signals or by using well known techniques such as the Nicholson-Ross-Weir method.

Electrical tissue properties can also be determined in the time domain. In these cases, the frequency domain microwave signal is transformed into a time domain signal. Discontinuities in the frequency domain data may be removed by applying a window, prior to the time domain transform. The aim of windowing is to improve the characteristics of the time domain signal. The window may include any known window type such as, but not limited to, flat top, Hamming, Hanning, Tukey or a low pass filter response such as, but not limited to, Butterworth, Chebyshev, elliptic.

In at least one example, after transforming the frequency-domain signal into a time-domain signal, electrical properties are determined by measuring time delay and/or amplitude of the time domain signal.

More generally, as described in the above-noted reference, the time delay is estimated as the peak of the time domain response, corresponding to the group time delay or is estimated from the leading edge of the time domain response, corresponding to the first break time delay. The time delays may be combined with separation between the arrays and speed of light in vacuum to estimate the permittivity. The amplitude of the time domain signal may be combined with separation between the arrays to estimate the conductivity.

In some embodiments, the method involves determining tissue response properties by using the difference between the parameters extracted from the measured conditioned microwave signal, and a reference microwave signal. The reference signal is measured with a material of known electrical properties and with the same separation distance and orientation between the antenna arrays. The reference material may consist of air, water, a water glycerin mixture, Triton-X water salt mixture, any engineered deformable material or liquid with known electrical properties.

In at least one example, selected measurements of the reference material may be collected and used to create a model that predicts parameters extracted from reference measurements in different scenarios. For example, time delays may be calculated from measurements of a reference material at different separation distances and a linear equation may then be fit to the time delays. This allows prediction of time delays for additional separation distances without the need for a specific reference measurement.

In some embodiments, the estimation method calculates the electrical properties by using the difference between microwave signals measured at different separation distances. This consists of subtracting the measured response at one separation distance from the response measured at a second separation distance, leading to a response corresponding to the compressed tissue without any other effects of the antenna or switching matrix.

In some embodiments the estimation method calculates the electrical properties using plane wave techniques to calculate permittivity and conductivity in the frequency domain.

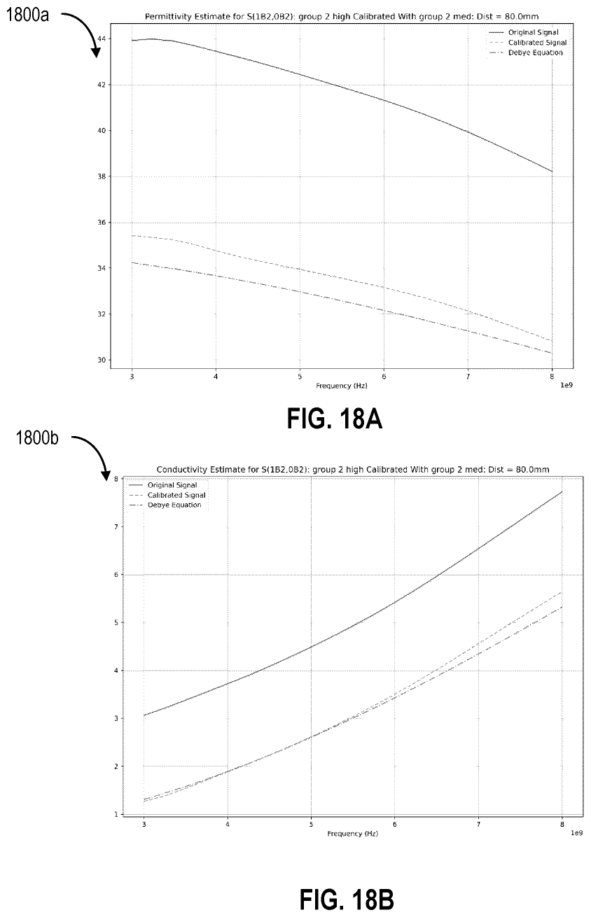

To this end, FIGS. 18A and 18B show plots (1800a), (1800b) for the final result of the correction for gain and phase center when the material properties are calculated with the Nicolson Ross technique.

### (iv.) Generating Output.

The following is a discussion of the outputs generated at (1314a) (FIG. 13A).

### (iv.a) Combining Tissue Response Properties

In some examples, the tissue response properties—estimated at multiple transmitting and receiving antenna pairs—are combined over the region of biological tissues scanned, to generate an output (e.g., output dataset).

In at least one example, the system can define a two-dimensional data collection plane (550) (FIG. 5B). The data collection plane (550) may be equally distal between the two arrays (550), and in some examples, along a parallel plane to the two arrays. The system can then determine the intersection of each signal path (508) with that plane (550), e.g., based on the known signal path properties of each signal. For example, this is shown as intersection point (552) for signal path (508′), in FIG. 5B. Accordingly, any tissue electrical properties—determined from a given signal path (508) travelling through the tissue—is mapped to the corresponding intersection point (552) on the plane (550). In this manner, each intersection point defines a “data point” (552), along the plane (550). This, in turn, generates an output 2D dataset characterizing the tissue response, along multiple data points on the 2D horizontal plane (550). In other examples, the plane (550) can be defined at any other distance or angle, relative to the two arrays.

To this end. if multiple tissue properties are determined at the same data point (552), along the plane (550) (e.g., because multiple signal paths intersect the same point), then these tissue properties can be associated separately with that point, or otherwise combined (e.g., average or mean of the tissue response property values).

In other examples, multiple planes can be defined, and the process repeated, such as to generate a 3D dataset, characterizing the tissue response in 3D and along different planes (see e.g., plane (550′) and intersection point (554). The 3D data set may represent a point cloud generated by discretization of the paths available within the volume of interest. To this end, an infinite number of planes can be theoretically defined, to generate 3D data with the desired resolution.

For each of the 2D and 3D dataset, interpolation can be used to generate filler tissue properties, in respect of any points, along any plane, that are not quantified by measured signal paths.

### (iv.b) Determining Tissue Features.

In some examples, the output can relate to determined tissue features, of the scanned biological tissue. Tissue features can include, for example, water content, water state (bound vs free), and ion density. These features can be determined by relating at least one or a combination of tissue electrical properties, for microwave signal paths, to particular tissue features.

For example, specific permittivity properties, of microwave signals, can be mapped to known water content ratios, correlated to the permeability properties. Accordingly, the tissue electrical properties can be converted (e.g., mapped to) tissue features, based on the known relationship between these tissue properties and tissue features.

In some examples, for the 2D or 3D output dataset, described above—each 2D or 3D output data point can be mapped to a respective tissue feature, for that datapoint. In this manner, a 2D or 3D tissue feature output is generated.

(iv.c) Generating 2D and/or 3D Images.

In some examples, the output corresponds to one or more images of the biological tissue, and is representative of the electrical properties and related tissues features. For example, this can involve generating a visualization of the 2D or 3D tissue property dataset, and/or 2D or 3D tissue feature dataset, as described above (see e.g., FIGS. 2A and 2B).

In some examples, the imaging is performed in real-time, or near real-time. Real time imaging may be implemented by limiting the number of frequency points recorded and/or increasing the measurement speed at a cost of reduced sensitivity. With adequate processing power the same algorithm presented herein may be used to reconstruct 2D images of breast properties. Alternatively, attenuation or phase shift value of the conditioned frequency signals, may be used to reconstruct qualitative 2D images of the breast.

### (iv.d) Output Relating to Characterizing Tissue.

In some examples, the microwave imaging device may implement a method to analyze images to characterize tissues, detect disease or response to treatment to obtain useful diagnostic information. For example, the analysis may comprise a step of comparing the images of the two breasts due to the expected symmetry in tissue distributions. If one breast exhibits a localized increase in properties, this may indicate breast disease.

In some embodiments, localized increases in images may also be used to define regions of interest in the image that may be tracked over time and/or compared with a similar region in the contralateral tissue. For example, comparison of an image over time may allow determination of a change in tissue properties which indicates whether or not a disease is growing or reducing over time, indicating the success or failure of a particular treatment which is being applied.

In some embodiments, the method to analyze images consists of identifying signals that exhibit different characteristics between or within the two breasts. For example, signals in a region of interest may be examined to identify differences in frequency content compared to signals in a region in the same breast or compared to signals in a similar region in the contralateral breast.

In some embodiments, the method to identify signals with different characteristics incorporates machine learning. This involves identifying characteristics of the time and/or frequency response of the measured signals that capture tissue response and features that are indicative of changes over time. By extracting these characteristics and features for a new set of measurements, a scan can be classified as consistent between breasts, or consistent within a breast over time, or changes can be identified.

In some embodiments, the average of the properties of the image may be calculated and related to tissue characteristics. For example, average properties of predominantly fatty tissues are expected to be lower than predominantly glandular tissues. By creating a model that maps average properties to tissue characteristics, density may be predicted from electrical property measurements.

### VI. Example Measurement Protocol

The following is a description of various protocols that can be followed while measuring, e.g., scanning, biological tissue using the microwave scanning system (400). This protocol can ensure consistent placement of the biological tissues while scanning is performed at different time points.

In at least one example, the measurement protocol consists of having the real-time imaging enabled while the operator places the biological tissues (412) within scanning region (158) (FIGS. 4A and 4B). The outline of the tissue location from previous images may be displayed, e.g., on display interface (114) (FIG. 1), with the real-time image to help the operator replicate similar placement.

In some embodiments, the measurement protocol consists of having the real-time feedback enabled while the operator places the biological tissues within the array. The outline of the tissue location from previous images may be displayed with an indication of sensors currently in contact with the tissues to help the operator replicate similar placement. The indication of contact may be obtained by analyzing measurements at a sub-set of or all frequency points.

The measurement protocol can also include automated feedback to the operator to guide repositioning of the biological tissues for best coverage or consistency of placement.

In at least one example, measurement protocol provides feedback on positioning of the tissue that is based on positioning of the contra-lateral side. For example, in case of breast imaging, if the right breast is scanned in the craniocaudal view, positioning information would be obtained from these scans to guide the subsequent scan of the left breast in the craniocaudal view.

In some embodiments, the measurement protocol ensures consistent separation distances (152) (FIGS. 4A and 4B), of the antenna arrays when measuring the same biological tissues at different time points. For this task, the system guides the operator through the user interface to replicate the separation distance from previous measurements. The guidance consists of displaying the separation distance of the previous measurement and instructing the operator to match that separation distance. The measurement protocol may include measurements at multiple separation distances (152), where the separation distance is modified by a small amount (relative to the overall separation) between measurements without repositioning the tissue.

The measurement protocol can also include multiple views of the tissue. The multiple views may be obtained by changing the angle of one or more arrays (102a), (102b), or by repositioning all arrays (102a), (102b). The multiple views that are collected are preferably in similar positions as other imaging devices. For example, the arrays (102a), (102b), may be positioned to replicate the craniocaudal and mediolateral-oblique views used in mammography.

In at least one embodiment, the measurement protocol includes similar or identical separation distances (152) when collecting the equivalent view of the contra-lateral tissue. As an example, if one breast is scanned in the craniocaudal view, then the separation distance(s) used for this breast are applied to the other breast of the same subject.

In some cases, the measurement protocol includes collecting multiple scans of the same tissue. This may be performed with or without repositioning the tissue.

### VII. Example Simulation Parameters

As discussed in FIGS. 13B and 13C, various simulations are performed to obtain reference correction factors, and more particularly, reference antenna response compensation factors.

In at least one example, the simulations of the antenna arrays with different biological tissues, to obtain correction and compensation factors, are performed using industry standard simulation tools. In at least one example, the simulation tools include Ansys™ HFSS™, CST™ or Sim4Life™.

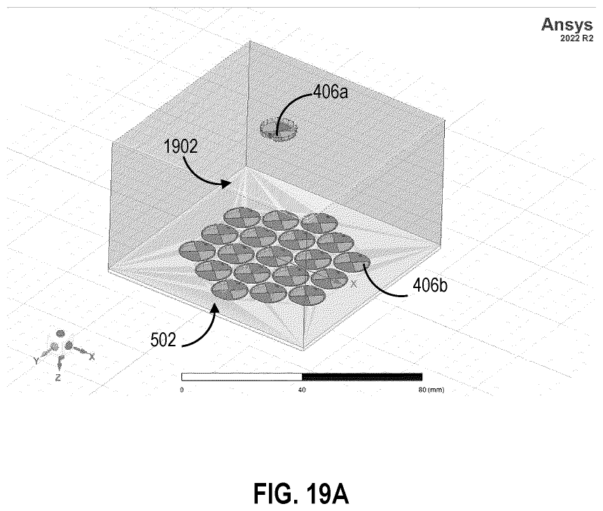

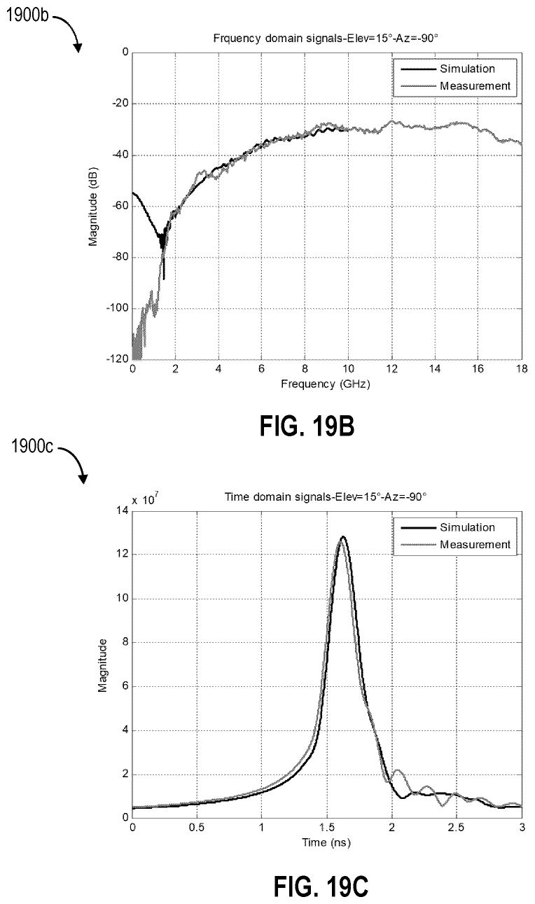

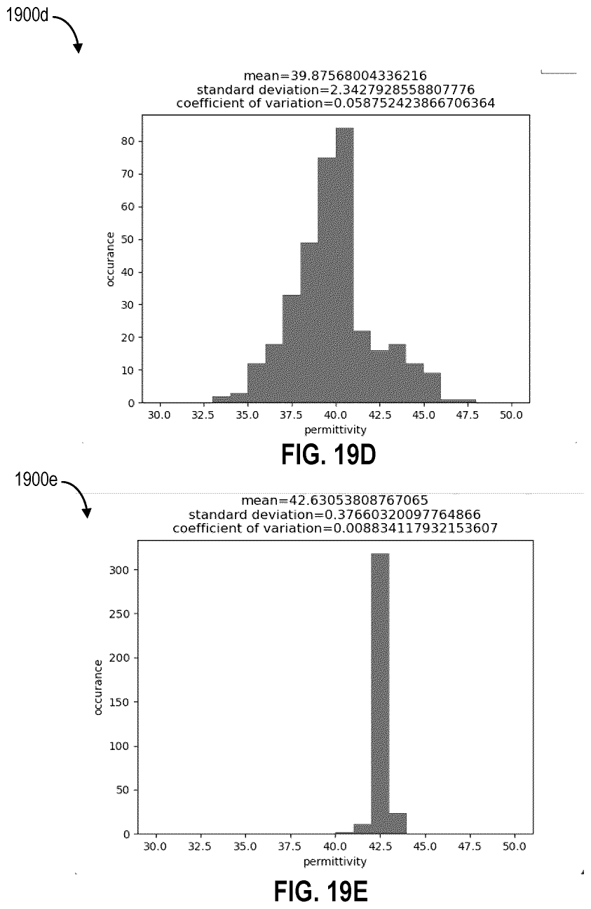

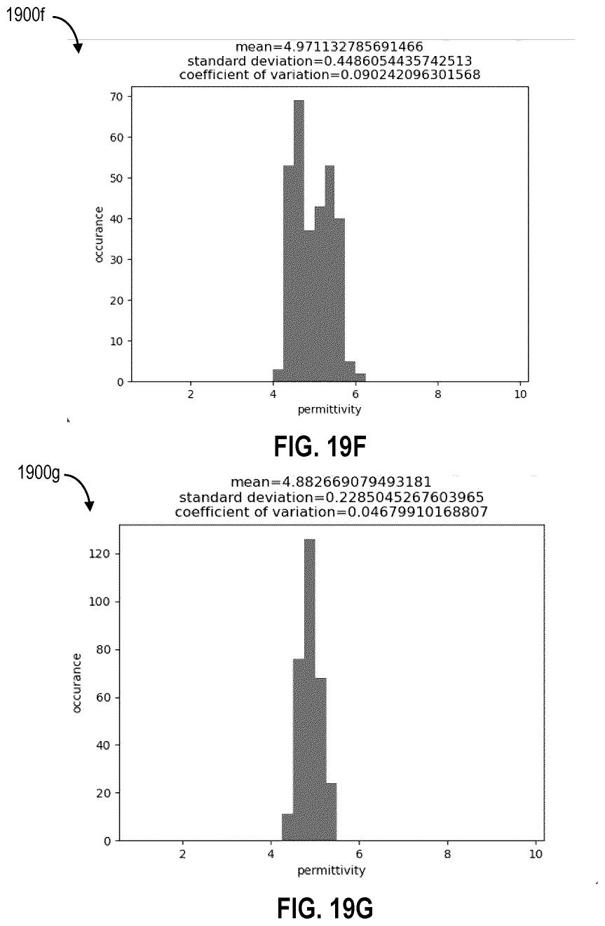

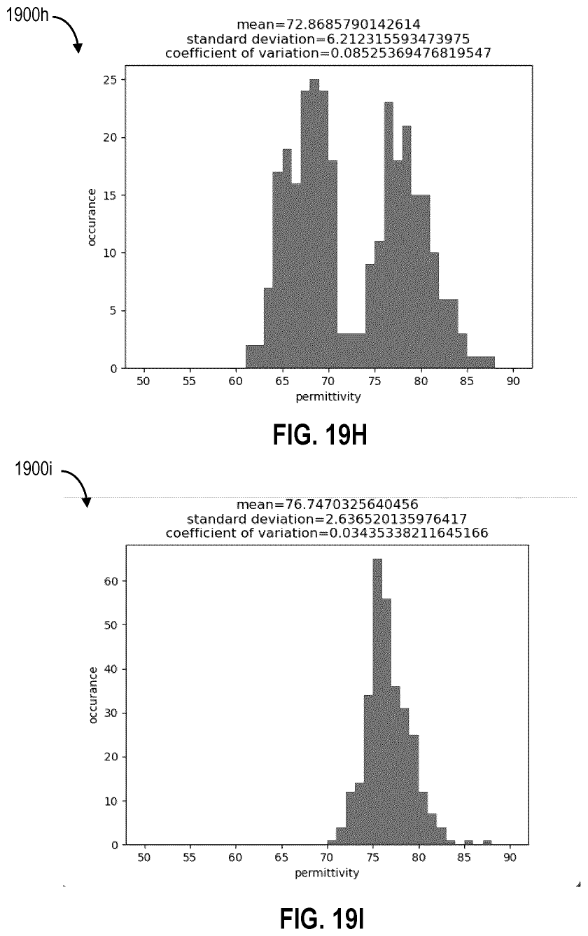

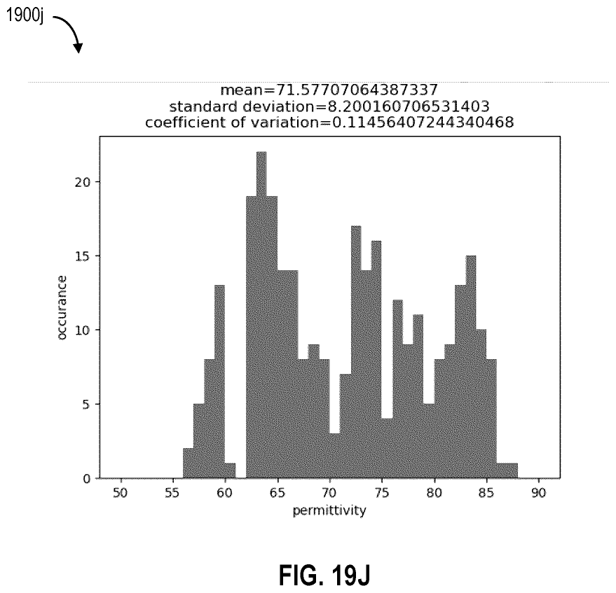

FIG. 19A illustrates an example image captured from an example simulation tool, and which shows an example simulated transmitting antenna element (406a), and a group (502) of receiving antenna elements (406b), in a simulated system (400). Region (1902) is filled with a simulated biological tissue.

In at least one example, the simulation is performed with a frequency sweep between 2 GHz to 8 GHz. Radiation boundaries are placed on the lateral faces of the biological tissues while the antennas are surrounded with perfect electric conductor (PEC), analogous to the actual array construction. The feeding is done directly through the feeding via (810) such that it captures all important antenna effects.

With respect to generating reference antenna response compensation factors—in one approach the transmission between a transmitting antenna (406a) and a set of receiving antenna (502) may be simulated while a tissue model is placed in region (1902) between the emitter and receivers. As noted above, various known Debye models—which model the permittivity and/or conductive properties of different biological tissue—are known, and are input into the simulation tool. For example, a Debye model for breast tissue is described in “Highly Accurate Debye Models for Normal and Malignant Breast Tissue Dielectric Properties at Microwave Frequencies, IEEE Microwave And Wireless Components Letters, Vol. 17, No. 12, December 2007, which is incorporated herein by reference.

In other examples, the biological tissue is simulated using known Cole-Cole models of each tissue. More generally, different models describing the range of tissues that may be encountered may be used to establish the antenna compensation factor. For example, tissue groups based on water content have been defined in M. Lazebnik, M. Okoniewski, J. H. Booske and S.C. Hagness, “Highly Accurate Debye Models for Normal and Malignant Breast Tissue Dielectric Properties at Microwave Frequencies,” in IEEE Microwave and Wireless Components Letters, 17, no. 12, pp. 822-824, December 2007, doi: 10.1109/LMWC.2007.910465, which is incorporated herein by reference. This reference defines “Groups 1, 2 and 3” tissues, having water content from high to low respectively.

To further expand the number of models simulating tissues, each tissue group may be further expanded with two additional models describing the low and high ranges with values that reflect the original data found in M. Lazebnik, M. McCartney, D. Popovic, C. B. Watkins, M. J. Lindstrom, J. Harter, S. Sewall, A. Magliocco, J. H. Booske, M. Okoniewski, and S. C. Hagness, “A large-scale study of the ultrawideband microwave dielectric properties of normal breast tissue obtained from reduction surgeries”, Phys Med Biol. 2007 May 21; 52(10):2637-56. doi: 10.1088/0031-9155/52/10/001. Epub 2007 Apr. 23, which is incorporated herein by reference.

To this end, in the simulated environment, the distribution of the receiving antenna set may follow the one of a specific antenna array (102). By simulating with different separation distances (152), the simulated model produces transmission data for a range of path directions that may be encountered during measurement. This transmission data is grouped using path directions characteristics, that are independent of the separation distance, such as the path azimuth (angular direction on the array plane) and path offset (lateral distance between the emitter and the receiver). Knowing the theoretical tissue response, the grouped transmission data are used to calculate the antenna compensation factors (gain and phase center) valid for that specific tissue model. Those antenna compensation factors are then fitted to a polynomial model that is a function of separation distance. By duplicating these simulation steps using multiple tissue models, antenna compensation factors can be defined for other tissue properties.

In at least one example, during a measurement, the tissue properties are first estimated to select the appropriate antenna compensation factors that are valid for a similar tissue property. Then the polynomial attached to the corresponding path azimuth and offset is selected to calculate the antenna compensation factor for the given separation distance.

To this end, a good match between measurement and simulation is necessary for the disclosed techniques to work. FIGS. 19B and 19C, which show plots (1900b) and (1900c), illustrate the level of agreement between measurement and simulations of two antennas transmitting microwave signals through a bag filled with tissue mimicking liquid (canola oil). As shown, frequency and time domain representations exhibit very good agreement, thereby confirming the validity of the simulation models employed in this disclosure.

FIGS. 19D-19F , which show plots (1900d)-(1900j), demonstrate the application of the disclosed techniques with a large amount of simulated data. The simulation contains one emitter and a set of 37 receivers separated with Group 1 low tissues (in accordance with the above cited references). The separation distance between the emitter and the receivers is swept between 40 and 80 mm with 5 mm step. This led to a data set containing 333 signals. The antenna response compensation is applied using different tissue properties (Tissue Group 1 Med) to mimic the case where the correction factor that matches the tissue group is not available.

In FIG. 19D, clearly observed is a spread of average estimates of permittivity when no correction is applied leading to a coefficient of variation of 0.058. FIG. 19E, shows a histogram plot of the calculated average permittivity when applying the antenna response compensation to the 333 simulated transmission signals going through the biological tissue model Group 1 low.

When the next the antenna response compensation is applied to measured data, a bag of glycerin material is placed between the two arrays and response is measured with a separation distance of 40 mm. FIG. 19F shows the average calculated permittivity using the raw signals. FIG. 19G shows the same data but with the antenna response compensation applied. The antenna response compensation factors simulated for Group 3 Med are used as they have the closest permittivity to glycerin. Comparing the results, the average permittivity remains constant however the coefficient of variation is reduced by a factor 2, demonstrating the effectiveness of the method.

In another example a bag filled with water is placed between the antenna array and measured at a separation distance of 40 mm. FIGS. 19H-19J show the calculated permittivity with the original signals, signals corrected using Group 1 Med factors and signals corrected using Group 3 Med factors, respectively.

The electrical property value of water is much closer to Group 1 Med as water average permittivity is at about 75 between over the frequency of interest while Group 1 Med has an average of 45. Group 3 Med on the other hand is quite a bit different with an average permittivity at around 5. In other words, the effect of no compensation is shown in FIG. 19F, the effect of compensation with fairly adequate factors is shown in FIG. 19I and the detrimental effect of compensating with inadequate factors is shown in FIG. 19J. The estimation becomes more accurate with adequate compensation with a mean permittivity value going from 72.8 to 76.7 and most importantly the coefficient of variation reduces from 0.085 to 0.034. It is also visibly a much tighter distribution. The contrary is true when inadequate compensation factors are used as it is clearly visible in FIG. 19J.

### VIII. Example Hardware Configuration for Controller

FIG. 20 shows an example electrical hardware configuration for the controller (418). As shown, the controller (418) can include the processor (2002) coupled to a memory (2004), as well as one or more of an input interface (2008), an output interface (2010), a communication interface (2006) and an input/output (I/O) interface (2012).

In some examples, memory (2004) can store various computer-executable instructions for performing the methods (1300a)-(1300c), or any portion thereof.

Input interface (2008) can include various devices for inputting data into the controller (418), e.g., keyboards, mouse, trackage, a virtual reality headset, gesture recognition, voice command recognition, or an augmented reality display. Input interface (2008) can be analogous to input interface (116) (FIG. 1).

Display interface (2010) can be an output interface for displaying data (e.g., an LCD screen). In some examples, the display interface (2010) comprises the display screen (114) (FIG. 1). In some examples, the display interface (2010) display a user interface that allows an operator to interact with the system.

In some examples, the input and display interfaces may be one of the same (e.g., touch display screen).

Communication interface (2006) may comprise a cellular modem and antenna for wireless transmission of data to the communications network. In some examples, where the above described methods are preformed using external computing devices (e.g., external servers), these external computing devices may communicate to receive and transmit data to controller (418), via the communication interface (2006).

I/O interface (2012) can be used to connect the controller (418) to other external devices, including the microwave transceiver (416) and the switching networks (402a), (402b).

To that end, it will be understood by those of skill in the art that references herein to controller (418) as carrying out a function or acting in a particular way imply that processor (2002) is executing instructions (e.g., a software program) stored in memory (2004) and possibly transmitting or receiving inputs and outputs via one or more interfaces.

### IX. Interpretation

Aspects of the present invention may be described with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The corresponding structures, materials, acts, and equivalents of all means or steps plus function elements in the claims appended to this specification are intended to include any structure, material, or act for performing the function in combination with other claimed elements as specifically claimed.

References in the specification to “some embodiments, “one embodiment”, “an embodiment”, etc., indicate that the embodiment described may include a particular aspect, feature, structure, or characteristic, but not every embodiment necessarily includes that aspect, feature, structure, or characteristic. Moreover, such phrases may, but do not necessarily, refer to the same embodiment referred to in other portions of the specification. Further, when a particular aspect, feature, structure, or characteristic is described in connection with an embodiment, it is within the knowledge of one skilled in the art to affect or connect such module, aspect, feature, structure, or characteristic with other embodiments, whether or not explicitly described. In other words, any module, element or feature may be combined with any other element or feature in different embodiments, unless there is an obvious or inherent incompatibility, or it is specifically excluded.

It is further noted that the claims may be drafted to exclude any optional or preferred element. As such, this statement is intended to serve as antecedent basis for the use of exclusive terminology, such as “solely,” “only,” and the like, in connection with the recitation of claim elements or use of a “negative” limitation. The terms “preferably,” “preferred,” “prefer,” “optionally,” “may,” and similar terms are used to indicate that an item, condition or step being referred to is an optional (not required) feature of the invention.

The singular forms “a,” “an,” and “the” include the plural reference unless the context clearly dictates otherwise. The term “and/or” means any one of the items, any combination of the items, or all of the items with which this term is associated. The phrase “one or more” is readily understood by one of skill in the art, particularly when read in context of its usage.

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values and ranges proximate to the recited range that are equivalent in terms of the functionality of the composition, or the embodiment.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc.

As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio.

1. A method for microwave scanning of biological tissue, comprising:
activating antenna elements, in an antenna set,
wherein the antenna set includes a transmitting antenna element in a transmitting antenna array, and one or more receiving antenna elements in a receiving antenna array;
operating at least one microwave transmitter to generate an interrogation microwave signal for transmission, via the transmitting antenna element, into a biological tissue;
receiving, via at least one microwave receiver, one or more received microwave signals from each of the one or more receiving antenna elements;
applying pre-conditioning to each of the received microwave signals to generate corresponding conditioned microwave signals, wherein the pre-conditioning isolates tissue response properties from extraneous response factors;
analyzing the conditioned microwave signals to determine one or more tissue response properties, associated with the biological tissue; and
generating an output based on the determined one or more tissue response properties.

2. The method of claim 1, wherein activating the antenna elements comprises operating a first switching network to couple the transmitting element to the at least one microwave transmitter, and operating a second switching network to couple the one or more receiving elements to the at least one microwave receiver.

3. The method of claim 2, wherein activating the antenna elements comprises operating the microwave transmitter coupled to the transmitting antenna element, and coupling a microwave receiver coupled to the receiving antenna element.

4. The method of claim 1, wherein the biological tissue is positioned in a scanning region between the transmitting and receiving antenna arrays, and contacts the antenna arrays.

5. The method of claim 4, wherein a dielectric cover material is coupled to one or more of transmitting and receiving antenna arrays, and contacts the biological tissue within the scanning region.

6. The method of claim 5, wherein the dielectric covering material has a relative permittivity below 5 and a maximum thickness of 2 mm.

7. The method of claim 1, wherein applying the pre-conditioning to a microwave signal comprises:
identifying a transmitting and receiving antenna pair, associated with the microwave signal;
determining one or more configuration parameters of the antenna arrays;
based on the determining, applying path-specific correction factors to the microwave signal to generate a corrected microwave signal; and
applying antenna response compensation factors to the corrected microwave signal to generate a conditioned microwave signal.

8. The method of claim 7, wherein the path-specific correction factors include correction factors for phase and magnitude correction for each the receiving pathway and transmission pathway associated with the transmitting and receiving antenna pair.

9. The method of claim 7, wherein the configuration parameters of antenna arrays correspond to one or more: (i) the positional spacing of antenna elements on each array; (ii) a spatial axial distance between the antenna arrays and/or (iii) a rotational orientation of the antenna.

10. The method of claim 9, wherein the antenna response compensation factors include antenna gain compensation factor and phase center compensation factors, and the antenna compensation factors are associated with: (i) the biological tissue type, and (ii) the configuration parameters of antenna arrays.

11. The method of claim 10, wherein the antenna response compensation factors and path-specific correction factors, are generated by simulation tools, which generate reference compensation and correction factors.

12. The method of claim 1, wherein each antenna array comprises a plurality of slot antenna elements, each slot antenna including a respective shielding interface.

13. The method of claim 2, wherein each switching network comprises one or more switching subnetworks, and each antenna array comprises one or more antenna subarrays.

14. A system for microwave scanning of biological tissue, comprising:
a transmitting and a receiving antenna array, each antenna array comprising a plurality of antenna elements,
wherein the antenna arrays are separated along an axis, by an axial separation distance, to define a scanning region for receiving biological tissue;
a first and a second switching network, each coupled to a respective transmitting and receiving antenna array, wherein the switching network comprises a plurality of switching elements;
at least one microwave transmitter coupled to the transmitting antenna array, via the first switching network;
at least one microwave receiver coupled to the receiving antenna array, via the second switching network; and
a controller coupled to the first and second switching network, and the at least one microwave transmitter and receiver, and operable to perform the methods of claim 1.

15. A system for microwave scanning of biological tissue, comprising:
a transmitting and a receiving antenna array, each antenna array comprising a plurality of antenna elements,
wherein the antenna arrays are separated along an axis, by an axial separation distance, to define a scanning region for receiving biological tissue;
at least one microwave transmitter coupled to the transmitting antenna array;
at least one microwave receiver coupled to the receiving antenna array; and
a controller coupled the at least one microwave transmitter and receiver, and operable to perform the methods of claim 1. 16. The system of claim 15, wherein the at least one microwave transmitter comprises a plurality of microwave transmitters, each microwave transmitter coupled to a separate antenna element of the transmitting antenna array, and
the at least one microwave receiver comprises a plurality of microwave receivers, each microwave receiver coupled to a separate antenna element of the receiving antenna array.

17. Antenna assembly for use with a system for microwave scanning of biological tissue, comprising:
at least two antenna arrays, wherein the antenna arrays are separated along an axis, by an axial separation distance, to define a scanning region for receiving biological tissue; and
a dielectric covering material coupled to and overlaying each antenna array, wherein the dielectric covering material contacts the biological tissue inserted in the imaging region defined.

18. The antenna assembly of claim 17, wherein each antenna array comprises a plurality of slot antennas.

19. The antenna assembly of claim 18, wherein each slot antenna includes a respective shielding interface.

20. The antenna assembly of claim 17, wherein the dielectric covering has a relative permittivity below 5 and a maximum thickness of 2 mm.
